(19) United States
(12) Patent Application Publication
(10) Pub. No.: US 2024/0062885 A1
(43) Pub. Date: Feb. 22, 2024

(54) SYSTEMS AND METHODS FOR GENERATING AN INTERACTIVE PATIENT DASHBOARD

(71) Applicant: Prenosis, Inc., Chicago, IL (US)

(72) Inventors: Jonah ELLMAN, Evanston, IL (US); Carlos G. LOPEZ-ESPINA, Evanston, IL (US); Bobby REDDY, Jr., Chicago, IL (US); Akhil BHARGAVA, Chicago, IL (US); lshan TANEJA, Cupertino, CA (US); Shah KHAN, Park Ridge, IL (US)

(21) Appl. No.: 18/260,764

(22) PCT Filed: Jan. 12, 2022
(86) PCT No.: PCT/US2022/012184

§ 371 Date: Jul. 7, 2023

Related U.S. Application Data
(60) Provisional application No. 63/136,580, filed on Jan. 12, 2021.

(51) Int. Cl. G16H 40/20; G16H 10/60; G16H 50/30

(52) U.S. Cl. CPC G16H 40/20; G16H 10/60; G16H 50/30

(57) ABSTRACT

A system for generating an interactive patient dashboard displaying metrics of a type of host response. The system may include processors and memory devices with instructions that configure the processors to perform operations. The operations may include transmitting a patient ID to a management platform or one or more devices (e.g., client devices), receiving electronic records, and employing a machine learning model to generate an acuity score based on the patient data. The operations may also include identifying critical parameters in the patient data by comparing parameters in the patient data with a distribution of parameters. The system can determine a ranking of the parameters, and generating a patient dashboard graphical user interface (GUI) for display. The dashboard GUI may include a prognostic indicator, and a list displaying the parameters according to the ranking.

### CROSS-REFERENCE TO RELATED APPLICATION

The present application is a 371 of International Application No. PCT/US2022/012184, filed Jan. 12, 2022, which claims priority to and the benefit of U.S. Provisional Patent Application No. 63/136,580, filed Jan. 12, 2021, titled “Systems and Methods for Generating an Interactive Patient Dashboard,” which are hereby incorporated by reference in their entirety as if fully set forth below and for all applicable purposes.

### TECHNICAL FIELD

The present disclosure generally relates to systems and methods for generating an interactive patient dashboard and, more particularly, to systems and methods for generating interactive graphical user interfaces for displaying information regarding the level of a host response for a patient, utilizing critical parameters and outcomes of machine learning models inputted with these parameters.

### BACKGROUND

A dashboard is a type of graphical user interface (GUI) which often provides at-a-glance views of indicators or other information relevant to a particular objective or business process. Dashboard interfaces may be used to display reports through different data visualization techniques. Further, dashboards GUIs often provide a platform to access other sources of information by, for example, providing links to disaggregated sources to facilitate user navigation.

Dashboard GUI's are particularly useful in settings where users need to analyze multiple sources of information and large datasets to make time sensitive decisions. For example, tasks that require interaction with multiple systems (e.g., to analyze disaggregated data) may use dashboard GUIs to centralize information and facilitate workflows.

Designing and implementing dashboard GUIs, however, has turned increasingly complex. While users need to consider more data from more sources (and in a shorter amount of time) the displaying devices are getting smaller and users have shorter attention spans. Thus, an effective dashboard should be designed to show useful information with simple visual representations of complicated data. And due to display space limits, effective dashboard GUIs need to intelligently select the most important data components, without seeming too cluttered to minimize distractions. These issues of dashboard GUI design have been exacerbated with the rise of machine learning, artificial intelligence, and big data analytics. These tools use complex algorithms to quickly analyze large datasets. But their output is often also complex and difficult to understand. Technologies such as machine learning are only useful if their outcomes can be presented to users in a way that allows simple and quick interactions to effectively facilitate decision making.

In light of the above, there is a need for systems and methods for machine learning models to consider multiple data and then provide actionable information as it relates to treatment and timetable. The described systems and methods herein address one or more of the problems set forth above and/or other problems in the prior art.

### SUMMARY

In various aspects, a system for generating an interactive dashboard graphical user interface (GUI) for displaying indicators based on one or more acuity values, associated with a risk category is described. In various embodiments, the system comprises one or more processors. In various embodiments, the system comprises one or more memory devices, wherein the one or more memory devices comprise instructions that, when executed by the processor, configure the one or more processors to perform operations comprising transmitting a patient ID to a management platform, receiving, from the management platform, at least one electronic record associated with a patient, the at least one electronic record comprising patient data, employing a machine learning model to generate an acuity score based on the patient data, the acuity score representing probability and level of a type of host response by the patient, employing a machine learning model to generate one or more prognostic values based on the patient data, the prognostic value representing a probability of an adverse event, determining a ranking of the at least one parameter according to an influence score associated with the at least one parameter, and generating a dashboard GUI for displaying, on or more client devices. In various embodiments, the dashboard GUI comprises or displays an acuity indicator displaying the acuity score and an associated risk category, at least one prognostic indicator displaying the at least one prognostic value and one or more risk categories associated with the at least one prognostic value, and a list displaying the parameters according to the ranking.

In various aspects, a computer implemented method for generating a dashboard GUI for displaying host response metrics is described. In various embodiments, the method comprises coupling an analytics server with a management platform through a FHIR API, generating a host response window embedded in an EMR, displaying an acuity indicator for presenting an acuity score on the host response window, wherein the acuity score is an output of a machine learning model and the acuity score from the machine learning model determines a probability and level of a type of host response based on patient data, displaying one or more prognostic indicators including one or more prognostic values on the host response window, wherein the prognostic value is an output of the machine learning model and the prognostic value from the machine learning model determines a probability of an adverse event, identifying, one or more critical parameters in the patient data by comparing the one or more parameters in the patient data with a distribution of corresponding one or more parameters of a target population, and displaying a list of the parameters according to a ranking based on influence scores associated with the one or more parameters, and displaying an emphasis indicator drawing attention to one of the parameters.

In various aspects, an apparatus is described. In various embodiments, the apparatus comprises one or more processors and one or more memory devices, wherein the one or more memory devices comprise instructions that, when executed by the processor, configure the one or more processors to perform operations. In various embodiments, operations comprise receiving, from a management platform, at least one electronic record associated with a patient, wherein the at least one electronic record comprises patient data, employing a machine learning model to generate an acuity score based on at least one of the parameters of the patient data, the acuity score representing probability and level a type of host response by the patient, employing a machine learning model to generate a prognostic value based on at least one of the parameters of the patient data, the prognostic value representing probability and level of an adverse event, identifying critical parameters in the patient data by comparing parameters in the patient data with a distribution of parameters of a target population, determining a ranking of the parameters according to an influence score associated with the critical parameters, and generating a dashboard GUI for displaying, on one or more client devices. In various embodiments, the dashboard GUI comprises an acuity indicator displaying the acuity score on the dashboard GUI and specifying a risk category, a prognostic indicator displaying the prognostic value on the dashboard GUI and specifying a risk category, and a list displaying the parameters according to the ranking.

### BRIEF DESCRIPTION OF THE DRAWINGS

The accompanying drawings, which are included to provide further understanding and are incorporated in and constitute a part of this specification, illustrate various embodiments and together with the description serve to explain the principles of the various embodiments. In the drawings:

FIG. 1 illustrates an exemplary architecture suitable for implementing machine learning methods, in accordance with various embodiments.

FIG. 2 illustrates a block diagram of an exemplary server and client in a machine learning system, according to various embodiments.

FIG. 3 illustrates a block diagram of an exemplary machine learning matching server, according to various embodiments.

FIG. 4 illustrates an exemplary process flow for generating a dashboard graphical user interface, in accordance with various embodiments.

FIG. 5 illustrates an exemplary process flow for authentication into the EMR, in accordance with various embodiments.

FIG. 6 illustrates a flow chart of an exemplary server process for generating an undesirable host response window in an Electronic Medical Record, according to various embodiments.

FIG. 7 illustrates a flow chart of an exemplary process for generating a machine learning model for determining the probability and severity of dysregulated and/or abnormal host response by a patient, according to various embodiments.

FIG. 8 illustrates a flow chart of an exemplary method for training a machine learning model using labels correlating to subtypes of undesirable (dysregulated and/or abnormal) host response, according to various embodiments.

FIG. 9 illustrates a flow chart of an exemplary process for determining an acuity score and/or a prognostic value, according to various embodiments.

FIG. 10 illustrates a flow chart of an exemplary process for identifying and displaying parameters (e.g., critical parameters) in patients on a parameter-by-parameter basis with respect to a reference population selected by the user, according to various embodiments.

FIG. 11 illustrates a flow chart of an exemplary process for identifying and displaying parameters (e.g., critical parameters) in an aggregated basis with respect a reference population selected by user the user, according to various embodiments.

FIG. 12A illustrates a flow chart of an exemplary process for generating a timetable of parameters used by a machine learning model, according to various embodiments.

FIG. 12B illustrates a flow chart of an exemplary process for generating a timetable of parameters used by a machine learning model, according to various embodiments.

FIG. 13 illustrates a flow chart of an exemplary timer generation and operation method with a summary status indicator displayed in a dashboard GUI, in accordance with various embodiments.

FIG. 14A illustrates a flow chart of an exemplary method for identifying local or global parameter importance used by a machine learning model, according to various embodiments.

FIG. 14B illustrates a summary box displaying parameters by ranking of influence based on a method such as that illustrated, for example, in FIG. 14A, in accordance with various embodiments.

FIG. 15A illustrates an exemplary dashboard GUI 1500 for dysregulated host response monitoring, in accordance with various embodiments.

FIG. 15B illustrates an exemplary dashboard GUI for dysregulated host response monitoring, in accordance with various embodiments.

FIG. 15C illustrates an exemplary dashboard GUI for dysregulated host response monitoring, in accordance with various embodiments.

FIG. 16 illustrates an exemplary dashboard GUI for undesirable host response monitoring, in accordance with disclosed embodiments.

FIGS. 17A, 17B, 17C, 17D, 17E, 17F, 17G, 17H, 17I, and 17J each illustrate exemplary dashboard GUI, or portion thereof, in accordance with various embodiments.

FIG. 18 illustrates an exemplary component of a dashboard GUI for identifying and displaying the value of parameters or parameters with respect to a reference population selected by user, according to various embodiments.

FIG. 19 illustrates exemplary dashboard GUIs (see e.g., 1910, 1920, or the like) for displaying local or global parameter importance ranking used by a machine learning model, according to various embodiments.

FIG. 20 illustrates an exemplary dashboard GUI for displaying local or global parameter importance contribution used by a machine learning model, according to various embodiments.

FIG. 21 illustrates an exemplary dashboard GUI for displaying local or global parameter importance contribution used by a machine learning model, according to various embodiments.

FIG. 22 is a block diagram illustrating an example software system utilizing the described systems, including the necessary components and interfacing, according to various embodiments.

FIG. 23 is a block diagram illustrating an example computer system with which the client and server of FIGS. 1 and 2, and the methods or GUIs of FIGS. 3-22 can be implemented, in accordance with various embodiments.

In the figures, elements and steps denoted by the same or similar reference numerals are associated with the same or similar elements and steps, unless indicated otherwise.

### DETAILED DESCRIPTION

In the following detailed description, numerous specific details are set forth to provide a full understanding of the present disclosure. It will be apparent, however, to one ordinarily skilled in the art, that the embodiments of the present disclosure may be practiced without some of these specific details. In other instances, well-known structures and techniques have not been shown in detail so as not to obscure the disclosure.

Developers of ML, artificial intelligence (AI), and neural network (NN) models often face the challenge of providing results efficiently through useful, concise, and actionable displays. Data visualization through a dashboard GUI can facilitate displaying results at the end of the machine learning process in a meaningful way to the end user. This is especially true in healthcare when trying to describe the complex health state of a patient.

A dashboard GUI may help healthcare professionals attempting to understand the health state of their patients accurately and rapidly, to decide on treatment pathways and/or to optimize care. One critical example of this in the understanding of the level of a type of host response (e.g., dysregulation and/or abnormality) of a patient's host response to external or internal stimuli. Various stimuli can cause a host response in patients, including but not limited to infections, immunotherapy, and trauma. Though many parameters are known to be associated with the level of abnormality of host response, such as temperature, cell counts, blood pressure, and others, there is currently no holistic and objective way for healthcare professionals to evaluate and understand the holistic abnormality for a given patient. In various embodiments, parameters can be stored in an electronic records stored on a management platform of a database.

Furthermore, there are too many parameters for a healthcare professional to (1) objectively compile the data together rapidly enough to use this information to make decisions to improve care for a patient and (2) visualize the most critical elements in a meaningful way in real time.

In various embodiments, healthcare professions can access the data described herein through a management platform (e.g., a database programmed to store healthcare related data). What is needed are systems and methods, described herein, for processing patient data using machine learning algorithms in such a way as to consider the parameters and provide actionable feedback (a treatment plan, including a timeline) or warnings (indicators showing the likelihood of a patient has or will have a condition and the likelihood of one or more adverse events occurring due to the condition.

One example of a scenario where a dashboard GUI can be helpful to a healthcare professional is a representation of the level of abnormality or dysregulation of host response due to a stimulus. This stimulus could include an infection, a therapy, trauma, and many others. Current techniques for the generation of GUIs provide no objective way for healthcare professionals to understand the holistic host response of a patient. Current GUIs fail to provide accurate visualizations and they are often unclear, failing to identify which patients with a potentially abnormal or dysregulated host response have the highest chance of deterioration and should be prioritized for care, or which care would be most appropriate for which patient.

ML or AI models that objectively input many relevant data parameter known to be representative of the level of abnormality or dysregulation of host response can provide a solution to this problem, however, the outputs of these models can be opaque and difficult to interpret for a human being. Because of this, these models are difficult and often impractical to use for healthcare professionals to make critical decisions that could dramatically affect patient outcomes. In order for healthcare professionals to effectively use these models, insight and intuition into the inner workings of the machine learning model for a specific patient's health state are necessary to build sufficient trust in a tool to rely on it to make critical patient care decisions.

Embodiments as disclosed herein provide a solution to the above problems in the form of systems and methods for generating dashboard GUIs that enable quick and intuitive display of complex machine-learning model outputs while providing critical insight to the user into the inner workings of the machine learning model for the current patient of interest. The described dashboard GUIs can accurately describe the level of abnormality and/or dysregulation of a patient's immune response to stimuli as outputted by a machine learning model. This level of abnormality and/or dysregulation has been shown to be correlated to a patient's chances for deterioration and potential to respond to specific treatments. The described embodiments can enable physicians to rapidly and precisely prioritize the appropriate care for the patients most likely to benefit from treatment at any given time. The disclosed systems and methods solve challenges in the technical field of machine-learning interactivity, providing tools that facilitate interpretation and manipulation of ML models, particularly applied to healthcare settings. Thus, in the context of healthcare, the disclosed systems and methods may facilitate improved outcomes for patients, such as reduced chance of mortality, readmission, high lengths of stay, high lengths of stay in Intensive Care Units, and other adverse events.

A benefit of the described techniques for dashboard GUI generation is a dramatic increase in the interpretability of the output of machine learning algorithms built to evaluate and output the level of abnormality and/or dysregulation of host response for a specific patient. These algorithms can input many parameters known to be correlated to the host response from the current patient and output an acuity score and/or prognostic value developed using the same parameter from many past patients in a training dataset. In various embodiments, the acuity score represents a probability of a patient having or developing a medical condition (e.g., sepsis). In various embodiments, the prognostic value(s) may include a probability of an adverse event occurring based on the acuity score. Non-limiting examples of adverse events may include change of death, change of escalation to ICU, or any other adverse events described herein or known to occur to patients. This can result in a much more objective and holistic representation of the level of abnormality and/or dysregulation of host response for the patient. However, a score outputted by an ML algorithm is often hard to understand for a healthcare professional user, leading to skepticism and lack of trust. As a result, many healthcare professionals choose not to use such ML algorithms. A dashboard GUI not only displaying an output score from a ML algorithm, but also clearly illustrating the parameters and methodologies that resulted in the score for the specific patient of interest, could build significant intuition and increase trust in the tool for healthcare professionals. This increase in intuition and trust is a critical necessity to find increased use in healthcare settings.

The disclosed systems and methods may also improve the technical field of automated generation of dashboard GUIs in health care settings. In particular, disclosed systems and methods may improve the technical field of generating dashboard GUI for small screen devices, such as mobile devices. In various embodiments, disclosed systems and methods may generate indicators that summarize complex machine-learning outputs so they can be displayed in small screens, while conveying actionable results to physicians or care providers. Further, the disclosed systems and methods may improve dashboard GUI by providing automated methods for categorizing, ranking, and selecting which information should be displayed to the user. For example, disclosed systems and methods may facilitate identifying critical parameters that influenced a machine-learning model output and generate plots or indicators (e.g., icons or text) for a summarized dashboard GUI. Therefore, the disclosed systems and methods solve problems of providing health care providers actionable and interpretable outputs from machine-learning models in a fashion that builds intuition and trust for the end user.

Moreover, disclosed systems and methods may generate specifically structured graphical user interfaces that improve accuracy and speed of interactions with healthcare data. For example, disclosed systems and methods may generate dashboard GUIs arranging patient information on a graphical user interface in a manner that assists healthcare professionals process information more quickly and accurately. Some embodiments of the disclosed systems and methods may generate dynamic GUIs that display machine learning outputs using dynamic lists, highlight icons/indicators, and/or graphical distributions to show specific parameters that are used during the machine learning process.

Reference will now be made to the accompanying drawings, which describe exemplary embodiments of the present disclosure.

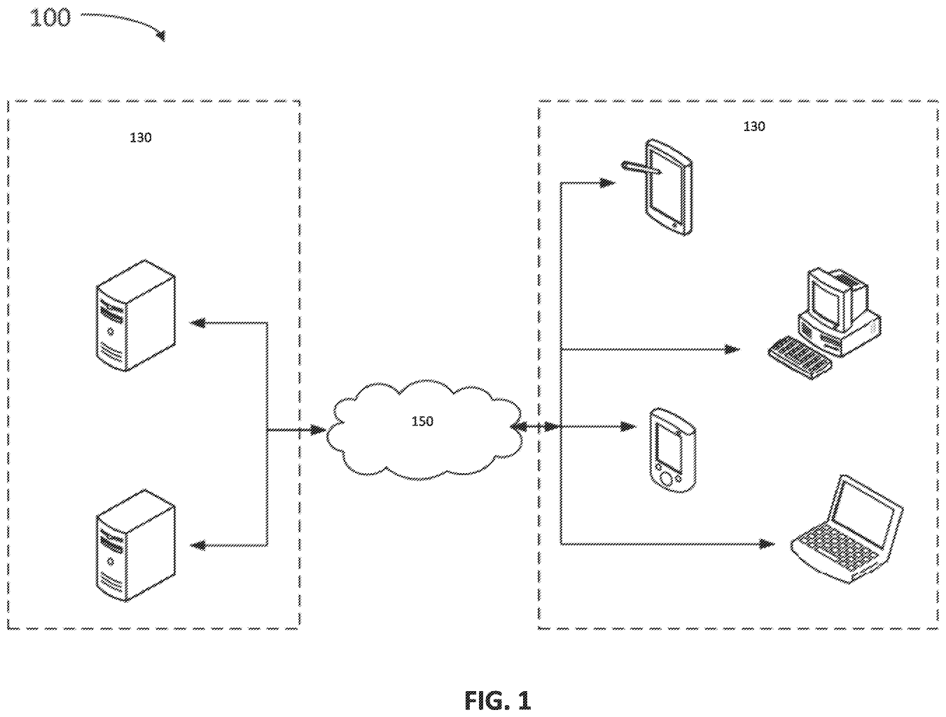

FIG. 1 illustrates a non-limiting example of an architecture 100 for implementing machine learning methods, in accordance with disclosed embodiments. Architecture 100 includes servers 130 and client devices 110 connected over a network 150. One of the many servers 130 is configured to host a memory including instructions which, when executed by a processor, cause the server 130 to perform at least some of the steps in methods and/or processes and logic flows as disclosed herein. At least one of servers 130 may include, or have access to, a database including clinical data for multiple patients.

In various embodiments, the database (e.g., one or more memory devices) may comprise instructions for performing a variety of operations. In some embodiments, the operations may include transmitting a patient ID from the database to a management platform. In various the operations may include receiving, from the management platform, at least one electronic record associated with the patient, the at least one electronic record including patient data. In various embodiments, the operations may include employing a machine learning model to generate a scores and values (e.g., an acuity score and/or one or more prognostic values) based on patient data. In various embodiments, the acuity score represents a probability and severity of a host response by a patient (e.g., to sepsis). In various embodiments, the operations may comprise identifying critical parameters in patient data by comparing one or more parameters in the patient data with a distribution of the one or more parameters of a reference (e.g., a target population). In various embodiments, the operations may include determining a ranking of the at least one parameter according to an influence score associated with the at least one parameter. In various embodiments, the database may include any of the data referenced herein.

In various embodiments, the operations may include generating a dashboard GUI for displaying, on or more client devices, a dashboard GUI comprising. In various embodiments, the dashboard may include an acuity indicator for displaying the acuity score. In various embodiments, the dashboard may comprise a prognostic indicator displaying the prognostic value and a risk category associated with the at least one prognostic value. In various embodiments, operations may include displaying a list of parameters according to a ranking.

Servers 130 may include any device having an appropriate processor, memory, and communications capability for hosting the collection of images and a data pipeline engine. The data pipeline engine may be accessible by various client devices 110 over network 150. Client devices 110 can be, for example, desktop computers, mobile computers, tablet computers (e.g., including e-book readers), mobile devices (e.g., a smartphone or PDA), or any other devices having appropriate processor, memory, and communications capabilities for accessing the data pipeline engine on one of servers 130. In accordance with various embodiments, client devices 110 may be used by healthcare professionals such as physicians, nurses or paramedics, accessing the data pipeline engine on one of servers 130 in a real-time emergency situation (e.g., in a hospital, clinic, ambulance, or any other public or residential environment). In some embodiments, one or more users of client devices 110 (e.g., nurses, paramedics, physicians, and other healthcare professionals) may provide clinical data to the data pipeline engine in one or more server 130, via network 150.

In accordance with various embodiments, one or more client devices 110 may provide the clinical data to server 130 automatically. For example, in some embodiments, client device 110 may be a blood testing unit in a clinic, configured to provide patient results to server 130 automatically, through a network connection. Network 150 can include, for example, any one or more of a local area network (LAN), a wide area network (WAN), the Internet, and the like. Further, network 150 can include, but is not limited to, any one or more of the following network topologies, including a bus network, a star network, a ring network, a mesh network, a star-bus network, tree or hierarchical network, and the like.

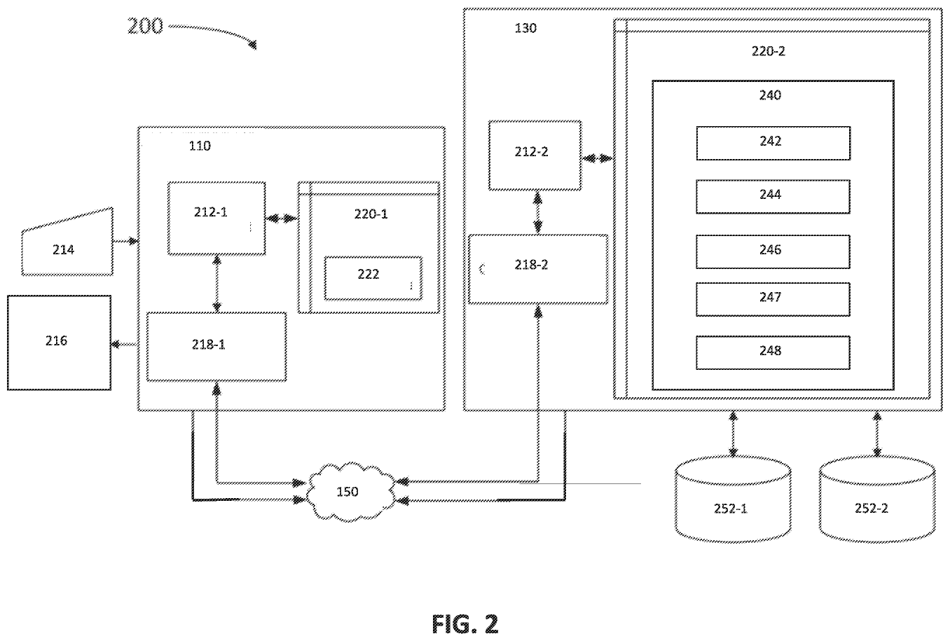

FIG. 2 is a block diagram 200 illustrating an example server 130 and client device 110 in the architecture 100 of FIG. 1, in accordance with various embodiments. Client device 110 and server 130 may be communicatively coupled over network 150 via respective communications modules 218-1 and 218-2 (hereinafter, collectively referred to as “communications modules 218”). Communications modules 218 are configured to interface with network 150 to send and receive information, such as data, requests, responses, and commands to other devices on the network. Communications modules 218 can be, for example, modems or Ethernet cards. Client device 110 and server 130 may include a memory 220-1 and 220-2 (hereinafter, collectively referred to as “memories 220”), and a processor 212-1 and 212-2 (hereinafter, collectively referred to as “processors 212”), respectively. Memories 220 may store instructions which, when executed by processors 212, cause either one of client device 110 or server 130 to perform one or more steps in methods as disclosed herein. Accordingly, processors 212 may be configured to execute instructions, such as instructions physically coded into processors 212, instructions received from software in memories 220, or a combination of both.

In accordance with various embodiments, server 130 may include, or be communicatively coupled to, a database 252-1 and a training database 252-2 (hereinafter, collectively referred to as “databases 252”). In one or more implementations, databases 252 may store clinical data for multiple patients. In accordance with various embodiments, training database 252-2 may be the same as database 252-1 or may be included therein. The clinical data in databases 252 may include metrology information such as non-identifying patient parameters, vital signs, blood measurements such as complete blood count (CBC), comprehensive metabolic panel (CMP), and blood gas (e.g., Oxygen, CO2, and the like), immunologic information, biomarkers, culture, and the like. The non-identifying patient characteristics may include age, gender, and general medical history, such as a chronic condition (e.g., diabetes, allergies, and the like). In various embodiments, the clinical data may also include actions taken by healthcare professionals in response to metrology information, such as therapeutic measures, medication administration events, dosages, and the like. In various embodiments, the clinical data may also include events and outcomes occurring in the patient's history (e.g., sepsis, stroke, cardiac arrest, shock, and the like). Although databases 252 are illustrated as separated from server 130, in certain aspects, databases 252 and data pipeline engine 240 can be hosted in the same server 130, and be accessible by any other server or client device in network 150.

Memory 220-2 in server 130 may include a data pipeline engine 240 for evaluating and processing input data from a healthcare facility to generate training datasets. Data pipeline engine 240 may include a modeling tool 242, a statistics tool 244, a data parsing tool 246, a data masking tool 247, and a similarity defining tool 248. Modeling tool 242 may include instructions and commands to collect relevant clinical data and evaluate a probable outcome. Modeling tool 242 may include commands and instructions from a linear model, an ensemble machine learning model such as random forest or a gradient boosting machine, and a neural network (NN), such as a deep neural network (DNN), a convolutional neural network (CNN), and the like. According to various embodiments, modeling tool 242 may include a machine learning algorithm, an artificial intelligence algorithm, or any combination thereof.

Statistics tool 244 evaluates prior data collected by a data pipeline engine 240, stored in databases 252, or provided by modeling tool 242. In some embodiments, statistics tool 244 may also define normalization functions or methods based on data requirements provided by modeling tool 242. Imputation tool 246 may provide modeling tool 242 with data inputs otherwise missing from a metrology information collected by data pipeline engine 240. Data parsing tool 246 may handle real-time data feeds and connect to external systems. Data parsing tool 246 may automatically label and characterize data optimized for efficiency and using group messages to reduce the overhead of the network. Data masking tool 247 may perform operations to create structurally similar but inauthentic version of healthcare records that, for example, remove personal identifiable information. Data masking tool 247 may be configured to protect the actual data while having a functional substitute for ML training Similarity defining tool 248, may perform operations for evaluating similarities between two datasets. For example, similarity defining tool 248 may employ comparative operations between clusters or vectors in two datasets like norms such as L2 norm, L1 norm, or other hybrid norms, or distance metrics such as Euclidean Distance, Manhattan Distance, Minkowski Distance or other distance metrics. Alternatively, or additionally, similarity defining tool 248 may be configured to extract parameter differences between datasets and/or identify similar and dissimilar records.

Client device 110 may access data pipeline engine 240 through an application 222 or a web browser installed in client device 110. Processor 212-1 may control the execution of application 222 in client device 110. In accordance with various embodiments, application 222 may include a user interface displayed for the user in an output device 216 of client device 110 (e.g., a graphical user interface, GUI). A user of client device 110 may use an input device 214 to enter input data as metrology information or to submit a query to data pipeline engine 240 via the user interface of application 222. In accordance with some embodiments, an input data may be sent to client devices in with an associated ranking of importance to enable validations and/or user review. Input device 214 may include a stylus, a mouse, a keyboard, a touch screen, a microphone, or any combination thereof. Output device 216 may also include a display, a headset, a speaker, an alarm or a siren, or any combination thereof.

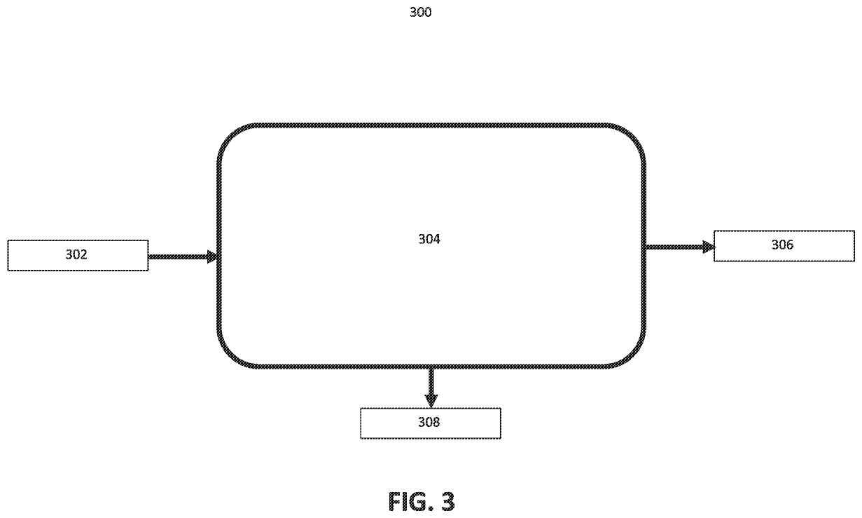

FIG. 3 illustrates a block diagram 300 of an exemplary machine learning matching server, according to disclosed embodiments. A set of input parameters 302 may be passed to a server 304 (e.g., a cloud server or intranet) for a given patient at a set point in time. Input parameters 302 may include any combination of parameters relevant to a host response (e.g., abnormal and/or dysregulated) due to stimuli, such as but not limited to vital measurements, demographic values, clinical lab results, blood biomarkers, urine biomarkers, saliva biomarkers, patient co-morbidities. Input parameters 302 could also include assemblies of any or all of these parameters, such as time trajectory values, combination parameters, and other transformations. Non-limiting examples of biomarkers may include plasma/serum protein markers, cell surface proteins, gene expression measurement, miRNA concentrations, cell counts, and other relevant biological parameters. Any combination of the input parameters 302 may be missing at any point in time when it is passed to the server 304 (e.g., a cloud server or intranet) in accordance with various embodiments.

The server 304 (e.g., a cloud server or intranet) may store a copy of the input/output data 308 from the internal machine learning algorithms on the server 300 to a history server in accordance with various embodiments. An outputs 306 of the server 304 (e.g., a cloud server or intranet) may include, for example, acuity score, prognostic value, a risk category, treatment guidance if any, a readiness flag, the parameters used for the outputs, and the influence score for each parameter, in accordance with various embodiments. The acuity score may be one of the main outputs of the machine learning algorithm, according to various embodiments. The acuity score and/or prognostic value(s) may represent the level of abnormality and/or dysregulation of the current patient's response to stimuli in accordance with some embodiments. The acuity may include a probability for a risk of contracting sepsis. One or more prognostic values may include a likelihood of an adverse event occurring to a patient (e.g., hospitalization within 24 hours). In various embodiments, an input parameter may include a parameter value. In various embodiments, the parameter values may be used to generate the acuity score, prognostic value(s), and to identify risk categories for a patient. In various embodiments, a risk category may be applied to a prognostic value. In various embodiments, a risk category may be determined by one or more parameter values. In various embodiments, a risk category displayed in a dashboard GUI may make it easier to select a treatment pathway for the patient. Guidance represents text fields that the algorithm or procedure could provide to optimize care for the patient. A readiness flag may represent a Boolean value true or false, corresponding to whether or not the output of the algorithm is ready to be displayed. In some embodiments, the acuity score and risk category may only be displayed if the readiness flag value is true. A list of parameters used 552 to generate the current outputted acuity and risk category may be displayed on a dashboard GUI. The influence score may be a representation of the importance of each parameter used to create the current outputted prognostic value and risk category, given the latest inputs (e.g., updated information can be added to change the values). This influence score can be used to rank the parameters, to emphasize and/or define an order in which the parameters are presented to the user to highlight the most important parameters for a given patient's health state at a specific point in time. The prognostic values represent the chance of adverse events (e.g. escalation to ICU, death, 30-readminssion, required vasopressor administration, required mechanical ventilation, etc.) within a particular period of time for similar patients, and can be used to inform care for the patient.

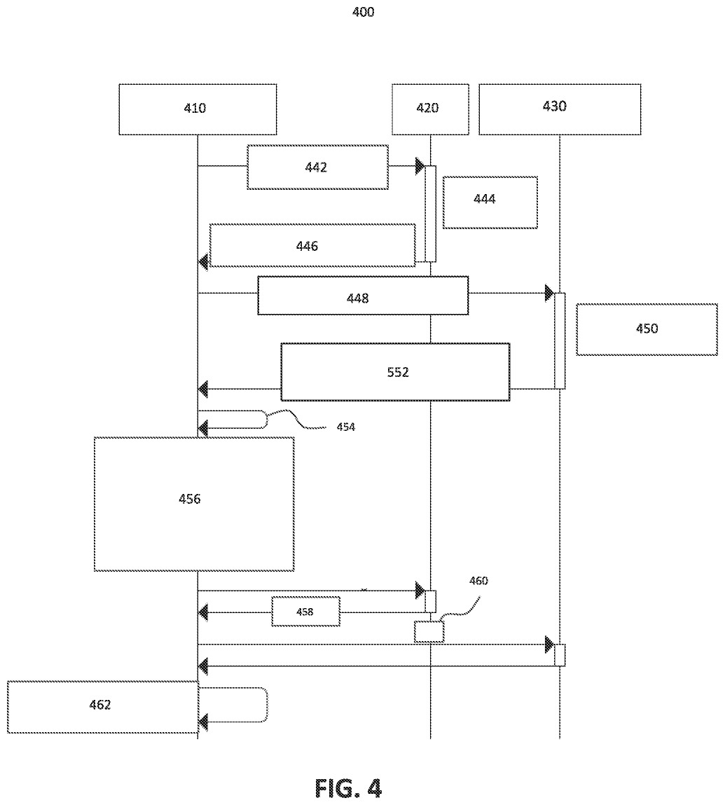

FIG. 4 illustrates an exemplary process flow 400 for generating a dashboard graphical user interface (GUI), in accordance with disclosed embodiments. Process flow 400 may be performed at least partially by any one of client devices coupled to one or more servers through a network (e.g., any one of servers 130 and any one of client devices 110, and network 150). For example, in accordance with various embodiments, the servers may host one or more medical devices or portable computer devices carried by medical or healthcare professionals. At least some of the steps in process flow 400 may be performed by a computer having a processor executing commands stored in a memory of the computer. In accordance with various embodiments, the user may activate an application in the client device to access, through the network, a data pipeline engine in the server (e.g., application 222 and data pipeline engine 240). The data pipeline engine may include a modeling tool, a statistics tool, a data parsing tool, a data masking tool, and a similarity tool (e.g., modeling tool 242, statistics tool 244, a data parsing tool 246, a data masking tool 247, and a similarity tool 248) to retrieve, supply, and process clinical data in real-time, and provide training data sets for forming ML models and/or a dashboard GUI.

A track server 410 first requests information about a patient by sending the patient the patient ID 442 to the EMR (electronic medical record) server 420 in accordance with various embodiments. The EMR server 420 responds with a variety of information about the patient. Non-limiting examples of patient information may include basic information about the patient and their stay in the hospital (their name, primary care provider, list of allergies, whether or not a blood culture has been ordered) and any combination of parameters relevant to abnormal and/or dysregulated host response due to stimuli, such as but not limited to vital measurements, demographic values, clinical lab results, blood biomarkers, urine biomarkers, saliva biomarkers, patient co-morbidities. A track server 410 may store this information so it can be displayed on the dashboard graphical user interface. In some embodiments, the communication with the EMR may occur using FHIR (Fast Healthcare Interoperability Resources) application programming interface 444 (API). In various embodiments, an order status 446 may be entered by a user and the algorithm may update an output and a dashboard GUI may be updated to reflect the output. In various embodiments, patient information 446 (e.g., clinical observations) may be entered by a user and the algorithm may update an output and a dashboard GUI may be updated to reflect the output.

The track server 410 can send a request to the match server 430 using the match API 450 with the data that was retrieved from the EMR server 420. The match server 430 may respond with acuity score, prognostic value, a risk category, treatment guidance if any, a readiness flag, the parameters used 552 for the outputs, and/or the influence score for each parameter. The data received from the match server 430 may be stored by the track server 410, in accordance with various embodiments. In many cases, the data may be displayed on the dashboard graphical user interface.

Based on the data obtained by the track server 410 from the EMR server 420 and the match server 430, the track server 410 can determine the patient status 456. This patient status can be updated using a patient status update model 454 for the patient over time by directly interacting with the track server 410. In some embodiments, the options for the patient status 456 could be used to track the workflow of a patient through different stages of being diagnosed and treated for sepsis and abnormal and/or dysregulated host response.

Track server 410 may be configured to periodically send more EMR server requests 458 to the EMR server 420 to fetch the most up-to-date information about the patient. This request could retrieve a similar kind of information as retrieved in the original request (e.g., patient ID 442) to the EMR server 420.

Additionally, or alternatively, track server 410 may be configured to periodically send more match server requests 460 to the match server 430 to fetch the most up-to-date acuity score, prognostic value, risk category, treatment guidance if any, readiness flag, parameters used for these outputs, and influence score for each parameter. For example, if new information was fetched from the EMR server 420, then this would change the information received from the match server 430.

The information described herein, may be aggregated and stored by the track server 410 about each patient can be available on the dashboard graphical user interface for easy consumption.

In some embodiments, a patient may remain on the dashboard graphical user interface until they are either discharged (e.g., patient removal 462 from dashboard GUI) from the hospital, they die, or they have been on the dashboard graphical user interface for more than 24 hours. At that point, the patient may be removed from the dashboard graphical user interface, so that only important relevant information is present on the dashboard graphical user interface.

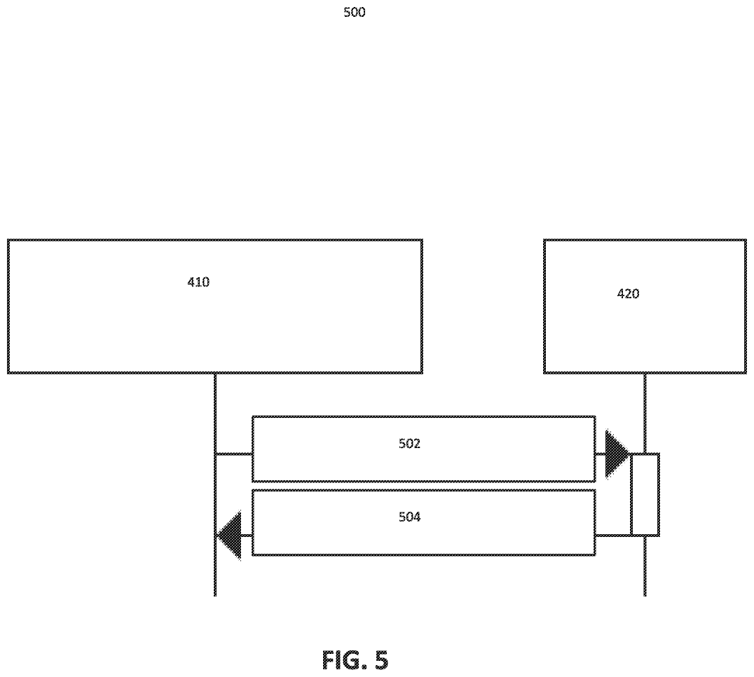

FIG. 5 illustrates an exemplary process flow 500 for authentication into the EMR, in accordance with disclosed embodiments. Process flow 500 may be performed at least partially by any one of servers or client devices coupled t through a network (e.g., any one of servers 130 and any one of client devices 110, and network 150).

The track server 410 may host a website including a dashboard graphical user interface. In various embodiments, a user may access the dashboard through the website and may need to be authenticated. In various embodiments, authentication may occur using an OAuth 2.0 protocol with the EMR server 420.

In various embodiments, a user may attempt to login into the track server 410 and be subsequently redirected to login 502 to the EMR server 420. If the login is successful, and the user grants access to the track server 410, then the user is redirected back to the track server 410 along with a FHIR (Fast Healthcare Interoperability Resources) access token 504 in accordance with various embodiments. The access token may be stored on the track server 410. In some embodiments, the stored token may be used to authenticate and authorize a user on the track server 410 during future login attempts and allow access to information on the EMR server 420. Access to information on the EMR server 420 may occur by using the FHIR (Fast Healthcare Interoperability Resources) API (Application Programming Interface) in some embodiments.

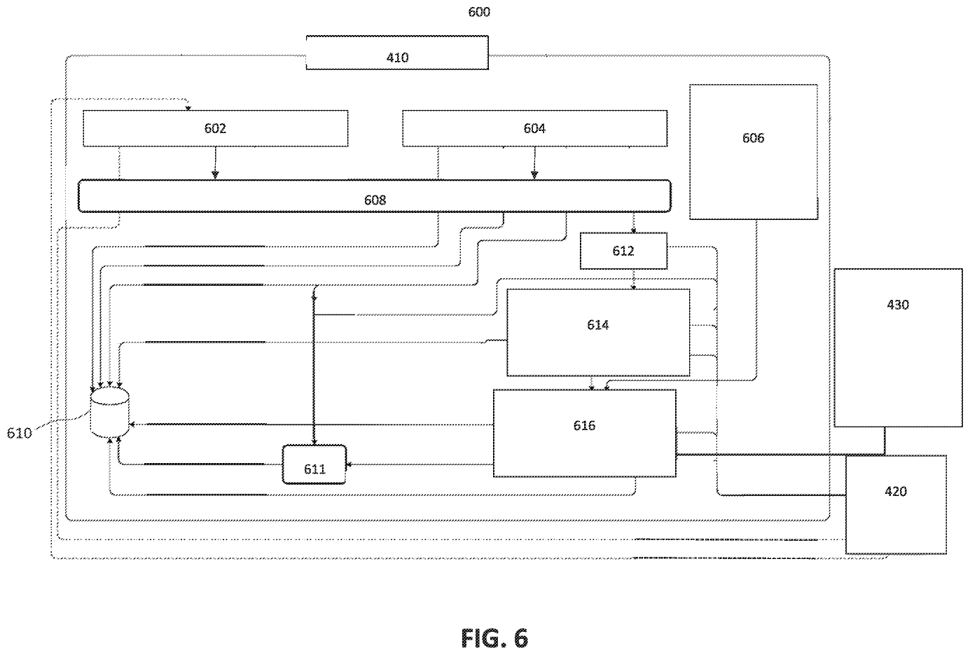

FIG. 6 illustrates a flow chart of an exemplary server method 600 for generating a dysregulated host response window in an EMR (e.g., for display on a dashboard GUI), according to disclosed embodiments. Method 600 may be performed at least partially by any one of servers or client devices coupled through a network (e.g., any one of servers 130 and any one of client devices 110, and network 150) in accordance with various embodiments.

The track server 410 may store a group of endpoints 604 used for authentication 602. In various embodiments, endpoints 604 can support allowing the user to login through the OAuth 2.0 protocol with the EMR server 420. The endpoints 604 may support storing session information after the user successfully logs-in using the OAuth 2.0 flow. The endpoints 604 may also support retrieving the session and logging out which deletes the session. Session data can be stored at the authentication layer 608.

The track server 410 can include a group of endpoints 604 for functionality for fetching (e.g., using a fetch patient module 612) and retrieving patient information (e.g., using a receiving patient information module 614 for onboarding purposes. Onboarding can also include registering a patient, collecting basic patient information (e.g., age, sex, etc.), and collecting and storing health related information. Patient information can be updated using a patient update module 616. Patient related data can be collected using any of the client devices described herein for onboarding and/or updating. Patient data can be saved using a save module 611, in various embodiments.

The endpoints 604 first pass through the authentication layer 608 which verifies the user is authenticated and authorized to access that endpoint. The endpoints 604 may also support fetching the list of patients to show on the dashboard graphical user interface, get details about a patient on the dashboard graphical user interface, registering a new patient on the dashboard graphical user interface, and/or updating the status of a patient on the dashboard graphical user interface, in accordance with various embodiments.

The track server 410 can make requests to the match server 430 to retrieve information like acuity score, prognostic value, risk category, treatment guidance if any, readiness flag, parameters used for these outputs, influence score for each parameter, and prognostic values. This information may be stored in database 610.

The track server 410 may make requests to the EMR server 420 to retrieve basic information about the patient and their stay in the hospital (their name, primary care provider, list of allergies, whether or not a blood culture has been ordered) and any combination of observations relevant to abnormal and/or dysregulated host response due to stimuli, such as but not limited to vital measurements, demographic values, clinical lab results, blood biomarkers, urine biomarkers, saliva biomarkers, patient co-morbidities. This information may be stored in database 610.

On a periodic basis (e.g., using a timing system 606), the track server 410 may automatically run an update patient module 616, which re-fetches information from the EMR server 420 and the match server 430 and stores this information in the database 610. This ensures that the information retrieved by the patient information for display on the dashboard graphical user interface is up to date.

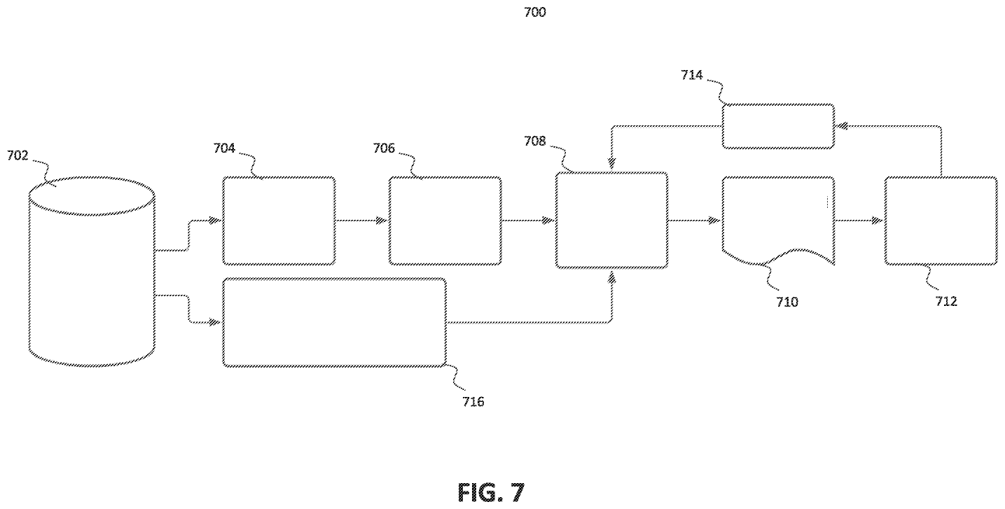

FIG. 7 illustrates a flow chart of an exemplary method 700 for training and validating a machine learning model for determining the probability of a response (e.g., a non-limiting example of a response includes dysregulation) of a patient, according to disclosed embodiments. Method 700 may be performed at least partially by any one of servers or client devices coupled through a network (e.g., any one of servers 130 and any one of client devices 110, and network 150). In various embodiments, the machine learning model may determine acuity scores. In various embodiments, the machine learning model may determine prognostic values.

A database 702 comprising past patient data, including but not limited to vital measurements, demographic information, lab results, co-morbidities, billing codes, physician notes, interventions, medications administered, patient outcomes, financial cost of care, and biomarker measurements from various sample matrices such as blood, plasma, serum, urine, and others. The data may include biomarker measurements not only measured at the time the patient was being cared for, but could also include measurements performed retrospectively on biobanked samples. For example, discards of plasma samples drawn for routine lab tests could be processed and frozen, be transported, and be stored in deep freezers. These samples could be thawed many months later and biomarker measurements could be performed from these samples. Using the time stamps of the original blood draws, this biomarker data could then be used as a representation of the patient's health state at a time point in the past. Using the database 702, parameters 704 up to a certain time point can be extracted. The parameter extraction 706 can be a combination of normalization, curation, imputation, and other methods.

The database 702 can also be utilized to generated dysregulated host response labels 716 that utilize some portion or all of the relevant patient data from the database 702. For example, a label for each patient of no sepsis or sepsis, may be defined as life-threatening organ dysfunction caused by a dysregulated host response due to infection, could be derived using a combination of lab results, physician notes, and medications administered. Using the labels 716, a machine learning model can be trained after undergoing model training 708 using various input parameters that have been extracted and the labels that have been defined from the dataset 702. Machine learning models can include linear models such as logistic regression or support vector machines, or non-linear models such as Random Forest or XGBoost models. The output of this training process can be a finalized machine learning model (e.g., model of label probability 710) that can input parameters and output a probability of a future patient being positive for the dysregulated host response label 716. This fixed model can be evaluated using a fresh bash of patients, the model can be adjusted, and it can be retrained until the final model is realized.

In various embodiments, the finalized machine learning model may undergo model evaluation 712 (e.g., validation). In various embodiments, a first set of patient data may be used to train a machine learning model (e.g., model training: label probability 708). In various embodiments, a second set of patient data may be used to validate the machine learning model (e.g., model evaluation 712).

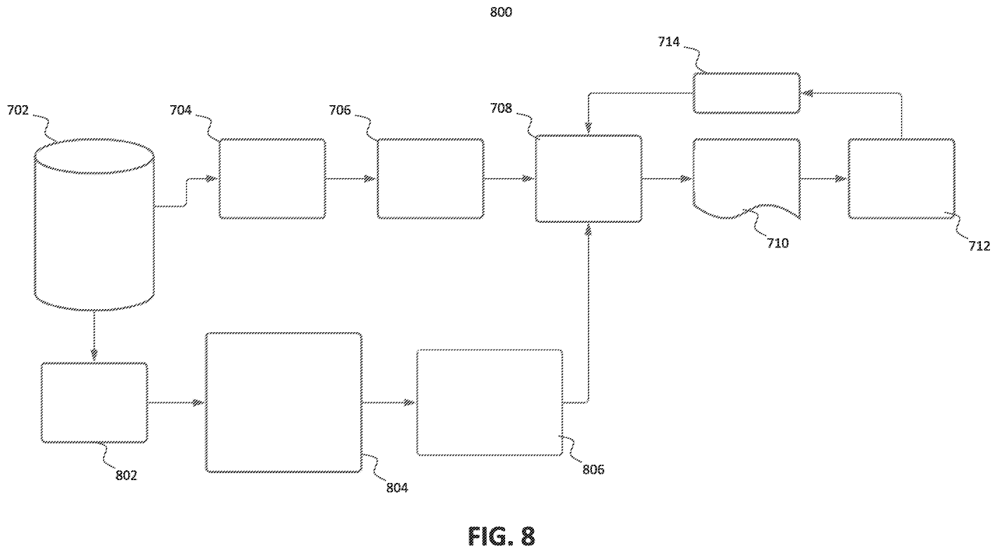

FIG. 8 illustrates a flow chart of an exemplary method 800 for training a machine learning model using labels correlating to subtypes of dysregulated host response, according to various embodiments. Method 800 may be performed at least partially by any one of servers or client devices coupled through a network (e.g., any one of servers 130 and any one of client devices 110, and network 150).

Alternatively or in combination with the approach of method 700, method 800 may include deriving labels of dysregulated host response 716. In various embodiments, parameter selection 802 can involve a user defined parameter selection and/or a machine learning derived method. In various embodiments, using an unsupervised method for patient label classification by dysregulated host response derived using some or all available patient data 804 can be utilized to define alternate collection of labels corresponding to dysregulated host response 806. For example, an unsupervised clustering technique may include inputting a series parameters, such as, biomarker measurements, vitals, labs, and demographic information could generate 10 clusters of different dysregulated host response due to infection, in some embodiments. Each subtype may have a different set of prognostic implications for patients and patients in each of subtype may benefit from different treatment plans. After collection of labels corresponding to dysregulated host response 806 has been defined using an unsupervised machine learning or statistical technique, the labels can be used to train a machine learning model (e.g., model of label probability 710) in a similar fashion to that described in method 700.

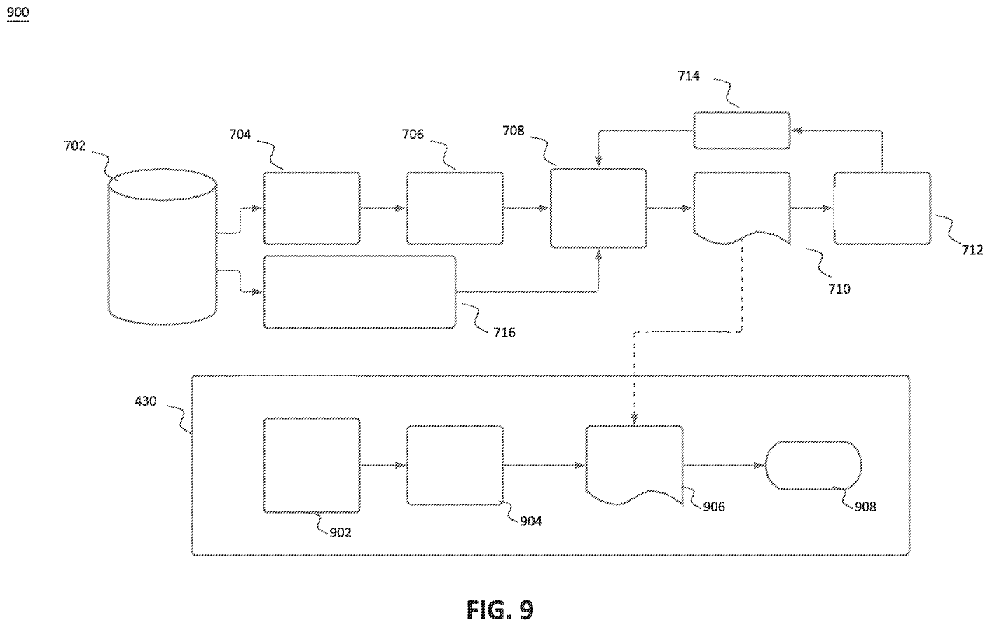

FIG. 9 illustrates a flow chart of an exemplary method 900 for determining an acuity score, according to various embodiments. In various embodiments, the method 900 can include determining one or more prognostic values. Method 900 may be performed at least partially by any one of servers or client devices coupled through a network (e.g., any one of servers 130 and any one of client devices 110, and network 150).

In various embodiments, once the machine learning model of label probability 710 is finalized, the machine learning model can be used to calculate a probability of a future live patient of having the dysregulated host response label 716. In various embodiments, a match server 430 can be used to carry out the calculation. The match server 430 can collect the relevant input parameters up to a certain time point (e.g., new live patient parameters up to a time point 902), some subset of parameters 704, for a new live patient. Parameter extraction 904, similar to parameter extraction 706, can be performed on these parameters to pass the parameters to the fixed model of label probability 710 and 906. Using the extracted parameters inputted into this model, an output of the new live patient's probability of exhibiting the dysregulated host response 716 can be generated, in accordance with various embodiments. The probability can either be utilized as probability itself 908, or a simple transformation could be executed to generate the score. For example, the probability from the new live patient could be compared to probabilities of the past patients to generate a percentile of how the likelihood of the label with respect to past patients, resulting in an acuity score 908 always between 0th to 100th percentile.

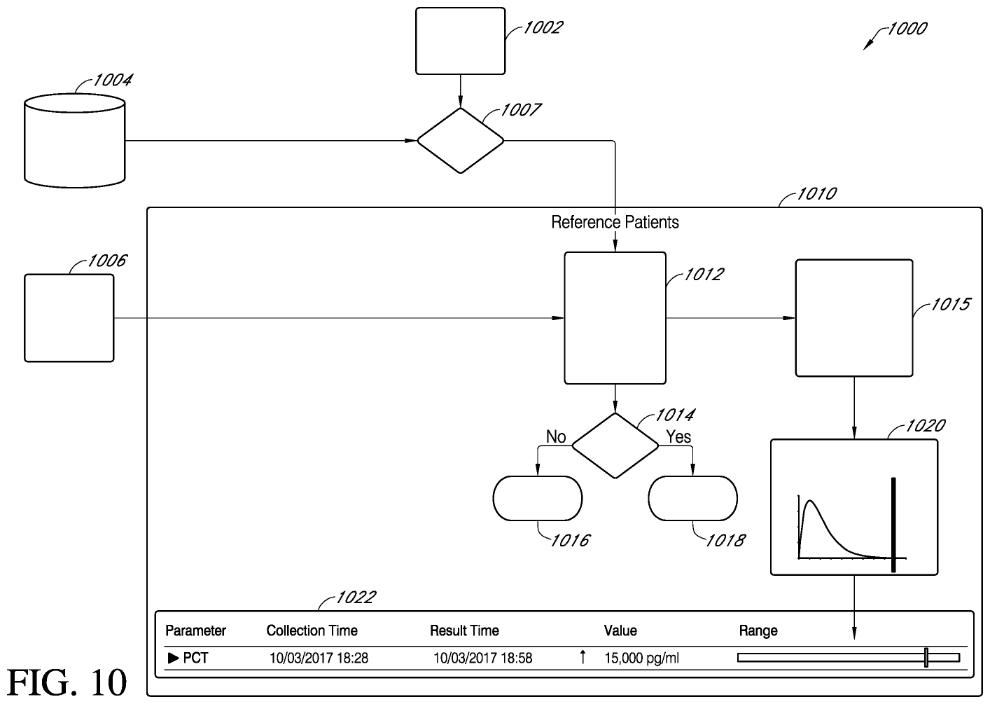

FIG. 10 illustrates a flow chart of an exemplary method 1000 for identifying and displaying parameters (e.g., critical parameters) in patients on a parameter-by-parameter basis with respect to a reference population selected by the user, according to various embodiments. Method 1000 may be performed at least partially by any one of servers or client devices coupled through a network (e.g., any one of servers 130 and any one of client devices 110, and network 150). In various embodiments, a system can carry the method 1000 in one or more steps. In various embodiments, the system 1000 may comprise a parameter analysis module 1010 and the parameter analysis module 1010 can include various processes (e.g., 1012, 1014, 1015, 1016, 1018, 1020, or 1022).

Using a target population 1002 that can be defined by a user such as a physician or other healthcare professional or a preset target population, a subset of patients can be selected from a comprehensive database of patients data full history 1004 (note in some embodiments, a database comprises a partial history) that includes many relevant parameters of dysregulated host response. The target population 1002 could be selected using a select patients module 1007 the dashboard GUI by the user with a simple drop-down menu. The target population may include many different combinations of patients, defined with single parameters (such as age, primary condition, single lab measurements, etc.) or with combinations of many parameters. The target population may also be defined with more sophisticated techniques, such as clustering analyses or density maps defined using unsupervised machine learning techniques that incorporate many potentially important input parameters.

For each parameter relevant to a given machine learning model, the parameter can be selected from a collection of parameters generated for a new live patient (e.g., new (live) patient parameters up to a time point 1006). The value for the parameter can be then be compared to a reference parameter of patients in the target population 1002 using a unidimensional statistical distance metric of individual patient versus reference patients 1012. Transformations to the values can be performed prior to calculating this unidimensional statistical distance metric. For example, z-score could be calculated for the parameter, and the unidimensional statistical distance metric could be inputted with this z-score instead of the raw measurement value. If the calculated distance is greater than a preset distance threshold 1014, the parameter for the specific new live patient can be considered an abnormal (e.g., critical parameter 1018). If the distance is not larger than the preset distance threshold 1014, the parameter for the specific new live patient can be considered a normal parameter 1016.

In various embodiments, the method 1000 can generate histogram distribution of distance reference patients vs individual patients 1015.

In some embodiments, when the relationship between the observed parameter for this specific new live patient and the parameter's distribution in the target population 1002 has been determined, a histogram 1020 depicting the distribution of the parameter in the target population 1002 and where the individual patient's parameter measurement lies within that distribution can be generated. The histogram can either be a two-dimensional density plot 1020, or a color bar chart. This histogram can be displayed in a graphical user interface, alongside the parameter name, relevant time stamps for that parameter, the raw value, and other reference symbols. This parameter line 1022 on the dashboard GUI could be used by a physician or other healthcare professional to quickly evaluate the value of the parameter and how critical the parameter is for the specific new live patient, compared to a relevant target population 1002. For example, it may be very informative to a physician to know how their 75-year patient's white blood cell count at a specific time compares to a distribution of 2000 past patients that are between 70-80 years old of age, instead of comparing the count to a general broad population. This parameter line 1022 on the graphical user interface can thus be critical to provide better insight and intuition into the state of a patient for a healthcare professional user.

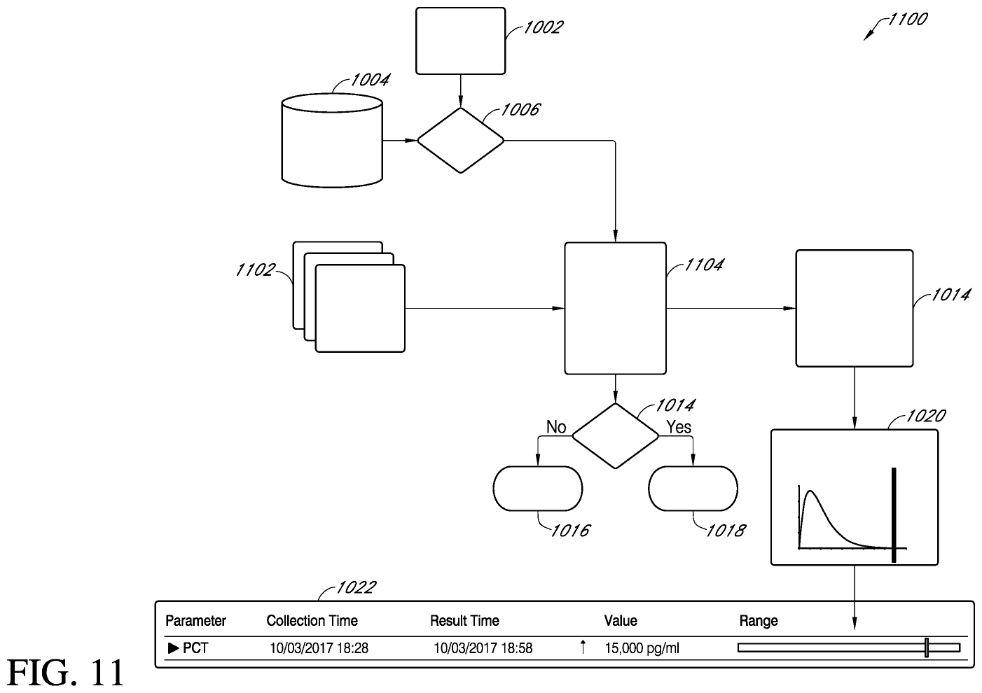

FIG. 11 illustrates a flow chart of an exemplary method 1100 for identifying and displaying parameters (e.g., critical parameters) in an aggregated basis with respect a reference population selected by user the user, according to various embodiments.

The method displays critical parameters with respect a reference population selected by user using multidimensional statistical techniques, according to disclosed embodiments. method 1100 may be performed at least partially by any one of servers or client devices coupled through a network (e.g., any one of servers 130 and any one of client devices 110, and network 150).

In accordance with various embodiments, method 1100 describes the use of many parameters at once to generate the target histogram, instead of just one parameter. In this case, several parameters up to a time point 1102 can be generated for a new live patient. These several parameters can be combined with the same parameters from the target population 1002, selected in the same fashion as method 1000, to construct a multidimensional statistical distance metric 1104. This metric 1104 can be constructed using a statistical technique such as Mahalanobis distance or other statistical methods to produce a single value representing the relationship between a new live patient's collection of parameter values and the same collection of parameter values from the past target population. A distribution of the past collection of parameters can be plotted as a histogram or density plot 1020, and can be displayed in a parameter line 1020, as described for method 1000.

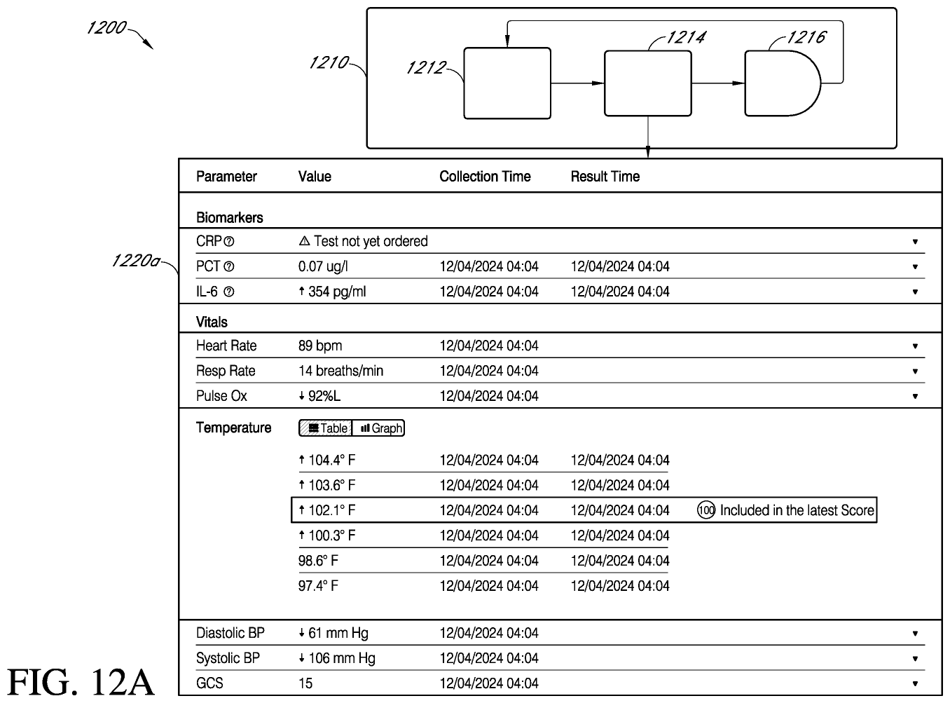

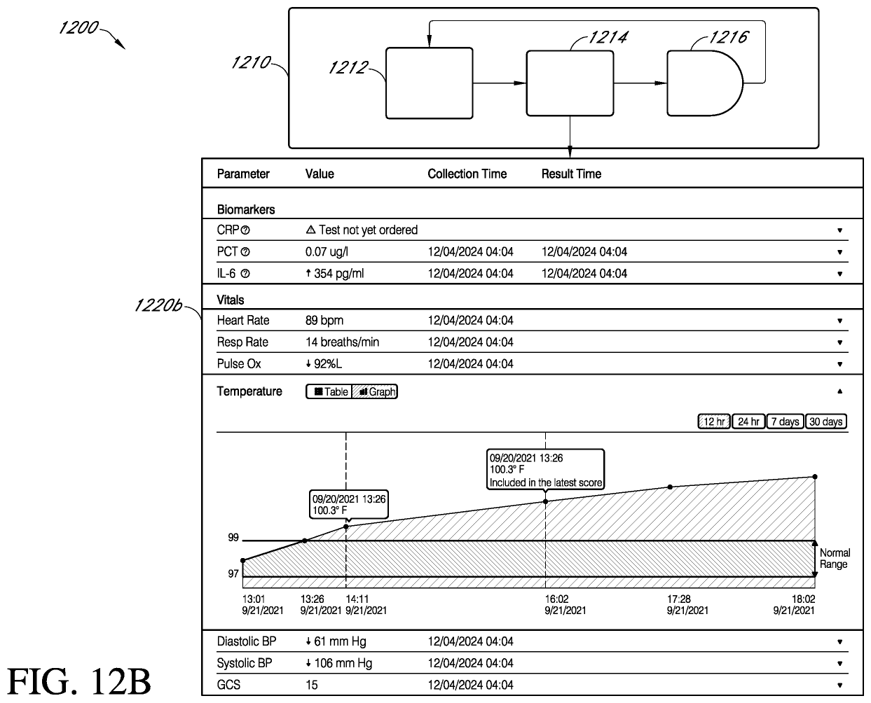

FIGS. 12A and 12B illustrate flow charts of exemplary methods 1200 for generating a timetable of parameters (see e.g., 1220a, 1220b, and the like) used by a machine learning model, according to various embodiments. Method 1200 may be performed on a system 1210 at least partially by any one of servers or client devices coupled through a network (e.g., any one of servers 130 and any one of client devices 110, and network 150), according to various embodiments. Parameter and time 1214 data can be stored on one or more databases described herein.

For each parameter to be displayed in dashboard GUI, a fetch from the EMR to grab the newest parameter values 1212 may be performed, according to some embodiments. The parameters, along with the corresponding time stamps for one or more activities. Non-limiting examples of activities include time of ordering, time of collection, time of result completion, and other relevant activities may be stored, according to various embodiments. A wait configurable time 1216, displays the wait time for the parameters being fetched. The parameters and their corresponding time stamps can be displayed in the graphical user interface display 1220, which includes many parameter information 1220 (e.g., parameter, value, collection time, and/or result time).

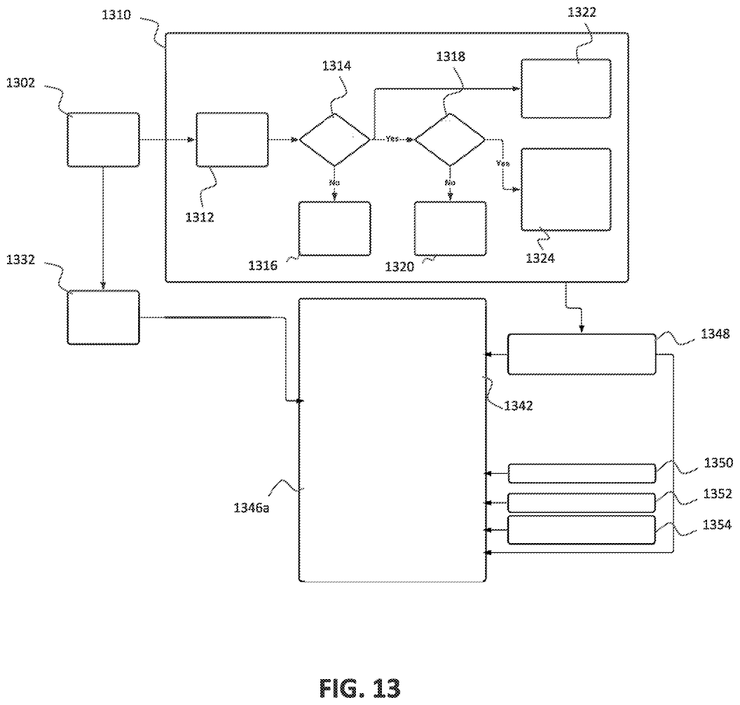

FIG. 13 illustrates a flow chart of an exemplary timer generation and operation method with a summary status indicator 1346a displayed on a dashboard GUI, in accordance with various embodiments.

Method 1300 may be performed at least partially by any one of servers or client devices coupled through a network (e.g., any one of servers 130 and any one of client devices 110, and network 150), in accordance with various embodiments. Step fetch patient time zero (e.g.: Triage) 1302 may include fetching a starting time (e.g., the time of triage or time of entering an emergency department), in accordance with various embodiments.

In various embodiments, one or more of the sub-steps of step 1310 may be performed for each treatment. In various embodiments, step 1310 may include step fetch patient treatment in bundle 1312.

In various embodiments, the method may incorporate one or more timers. The is the point that serves as the start of the treatment timers 1332. These timers may include one or more of set triage timer, treatment order timers 1322 (e.g., for each treatment) and treatment administered timers (e.g., for each treatment). For each treatment in a predefined bundle of treatments, a check 1314 can be performed to ascertain whether or not the treatment has yet been ordered. If the treatment has not been ordered, then the treatment order timers are updated 1316. If the treatment has been ordered, then the treatment may be identified as ordered and the treatment order timer is stopped. In addition, a check 1318 may be performed to see whether the treatment has been provided. If the treatment has not been administered, then an update to the treatment administration timers 1320 may be performed. If the treatment has been administered, the treatment is then identified as administered and the treatment administration timer is stopped 1324.

By collating the treatment ordered timer and treatment administered timers for the relevant treatments in a pre-specified bundle, an evaluation is periodically performed to check completion the treatments in the bundle 1348. If the treatments have not been completed, a timer displaying the time left before the healthcare team may be in violation of a preset protocol and can be displayed in a box 1342 on the dashboard GUI. For example, if the elements of a sepsis bundle as defined by the Sep-3 center for Medicare and Medicaid Services (CMS) have not been complete, the time left to complete these items before being in violation could be displayed as a countdown from 3 or 6 hours. In addition, the time from time zero can be displayed in box 1342 in the GUI. Finally, the treatment order and treatment administered timers can be used to check the status of a treatment in the hospital's workflow. This status can be displayed using a combination of check marks, alert signs, or ‘X’ marks in the GUI for the evaluation of the order of the treatments 1350, approval of treatments 1352, and/or administration of treatments 1354. A summary of this status 1346a can be displayed in the box 1342 in the GUI in accordance with various embodiments. A timer counting down from triage may be displayed in the box 1342, in accordance with various embodiments. A timer including multiple timepoints (e.g., timepoints for treatment order, pharmacy approval, administration, and completion of treatment, may be included in various embodiments. One or more summary status indicators may be displayed on the dashboard GUI, in accordance with various embodiments.

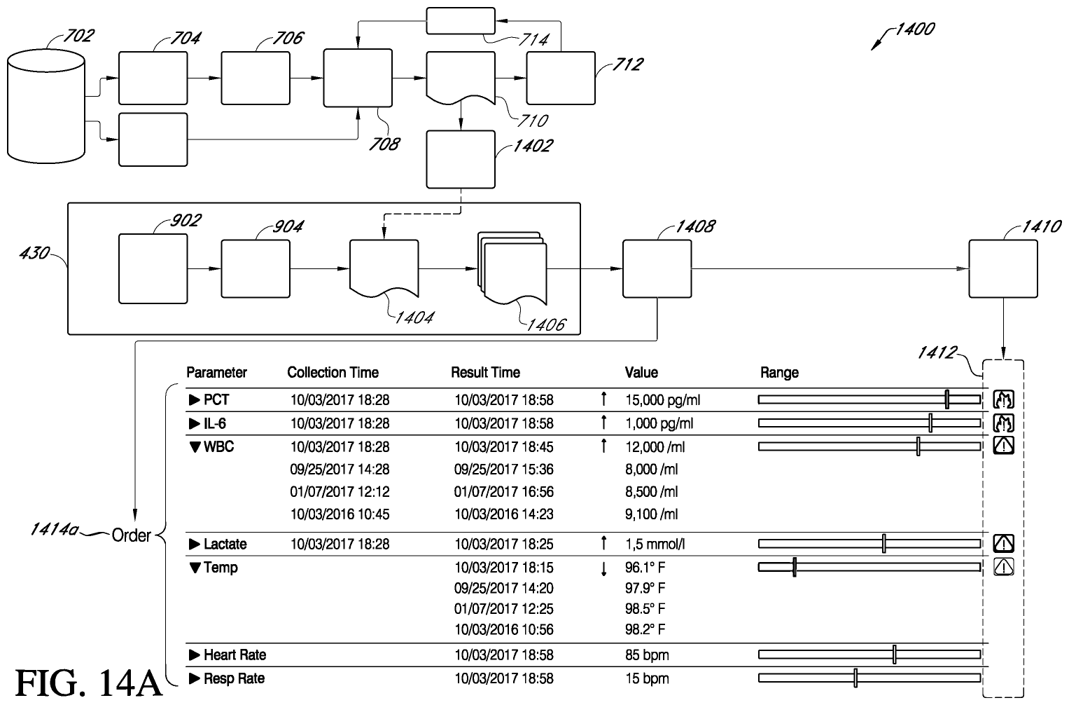

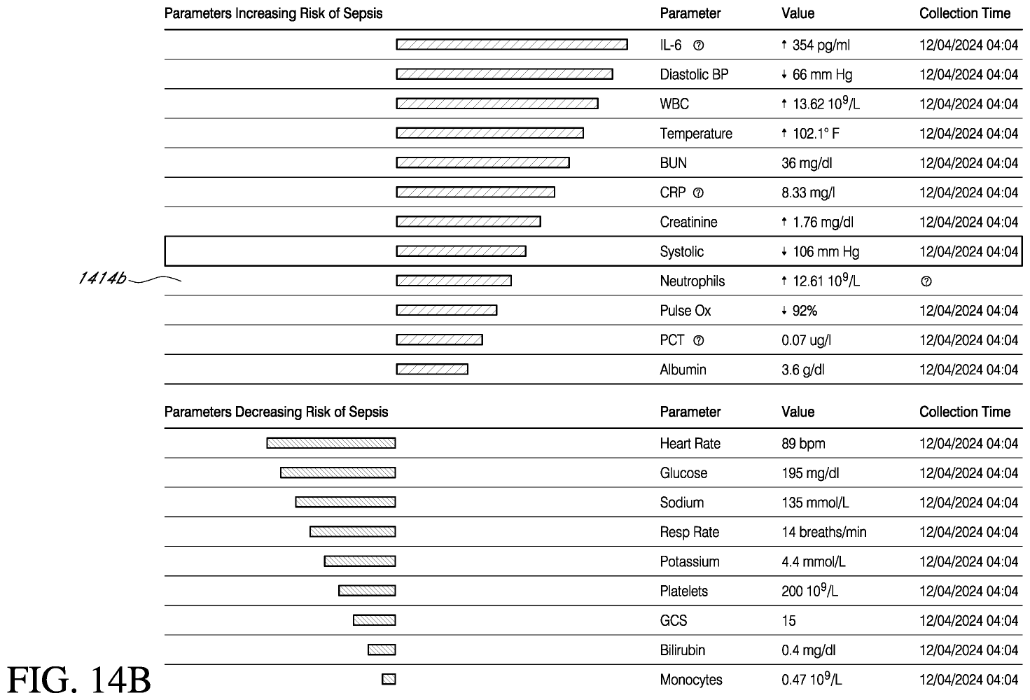

FIG. 14A illustrates a flow chart of an exemplary method 1400 for identifying and displaying local or global parameter importance used by a machine learning model, according to various embodiments. Method 1400 may be performed at least partially by any one of servers or client devices coupled through a network (e.g., any one of servers 130 and any one of client devices 110, and network 150).

Using methods 700 or 800, a machine learning model of label probability 710 model can be identified, in accordance with various embodiments. A model 1402 of parameter importance may be selected that matches the machine learning model (e.g., model of label probability 710). The model 1402 may be selected on a global level, meaning that one set of parameter importance values can be determined for a given model, in accordance with various embodiments. For example, for a model evaluating a host response (e.g., abnormality of host response), interleukin-6 may have the highest parameter importance, followed by procalcitonin, white blood cell count, and temperature at a global level for the patients in general. The parameter importance model 1402 may be based on coefficients of a linear machine learning model such as logistic regression or support vector machine, based on tree importance in a machine model such as random forest, or based on techniques used to evaluate parameter importance for other commonly used machine learning models, in accordance with various embodiments.

With a selected parameter importance model 1402, parameters up to a certain point for a new live patient parameters up to a time point 902 may be generated. After parameter extraction 904, this set of values is passed to an importance method 1404. This importance method incorporates at least two inputs: (1) the global model 1402 parameter importance and (2) the set of input extracted parameter values for a single patient at a single point in time. Using these inputs, the importance method 1404 generates an influence score 1410 for each parameter for the particular patient at this point in time, and at a local level. This can be very different for two different patients with different input parameter, even with the same machine learning model. For example, global parameter importance for a model for of host response (e.g., abnormal host response) could be ranked, from highest to lowest, as interleukin-6, procalcitonin, white blood cell count, and temperature. For Patient 1 with a very elevated temperature but normal interleukin-6, importance method 1404 may generate influence scores 1410 that rank the parameters at a local level for this patient from highest to lowest as temperature, procalcitonin, interleukin-6, followed by white blood cell count. In contrast, for Patient 2 with very abnormal interleukin-6 values and normal other values, though using the same machine learning model, the importance method 1404 may generate influence scores 1410 that rank the parameters at a local level as interleukin-6, temperature, white blood cell count, and procalcitonin.

The importance method 1404 can include simple calculations incorporating both the global parameter importance to the model and level of abnormality of each of the input parameter observed for the specific patient at a specific point in time. For example, the z-score of a particular observed parameter with respect to all or a subset of past patients in a database 702 could be simply multiplied by a quantitative measure of global parameter importance, and this value could be used as an influence score per parameter 1406 to rank parameters by importance at the local level. Alternatively, more sophisticated methods such as Mahalanobis distance or Shapley Additive Explanation techniques could be used to generate the influence score per parameter 1406. However, the output of the importance method 1404 must be a parameter influence score 1406 that may be generated for each parameter used as input into the model of label probability 710.

After the influence score per parameter 1406 has been generated for each parameter for a particular observation, a ranking of the influence score per parameter 1406 can be performed, in accordance with various embodiments. After ranking, the parameters can be displayed in the final GUI in a summary box 1414a having many parameter lines in order of decreasing or increasing parameter importance for the given patient.

FIG. 14B illustrates a summary box displaying parameters by ranking of influence based on a method such as that illustrated, for example, in FIG. 14A, in accordance with various embodiments. For example, the summary box 1414b may include a list of parameters increasing the risk of sepsis as described herein, in accordance with various embodiments. In various embodiments, the relative severity may be displayed in bar format in the summary box 1414b. In various embodiments, parameters may be displayed in addition to associated values and collection times as described herein.

The summary box 1414b can be to provide intuition and insight for a healthcare professional user of the GUI into how the output (e.g., acuity score and/or prognostic value) of a machine learning model was calculated. In various cases, parameters may be associated with a risk scale (e.g., a graphical presentation of relative impact of a given parameter on an acuity score). This could increase trust in the overall model and chances that a healthcare professional will act upon the information. In addition, symbols or numbers describing the influence score for each parameter 1406 on the GUI could be included in a side panel 1412 if the user wishes to see quantitative evaluations of this importance. In various embodiments, an influence score may be represented by a graphic or chart. For sake of clarity, the order displayed in the summary box 1414a, 1414b may change for each specific patient observation at each point in time, depending, on the influence scores per parameter 1406 outputted for that particular observation.

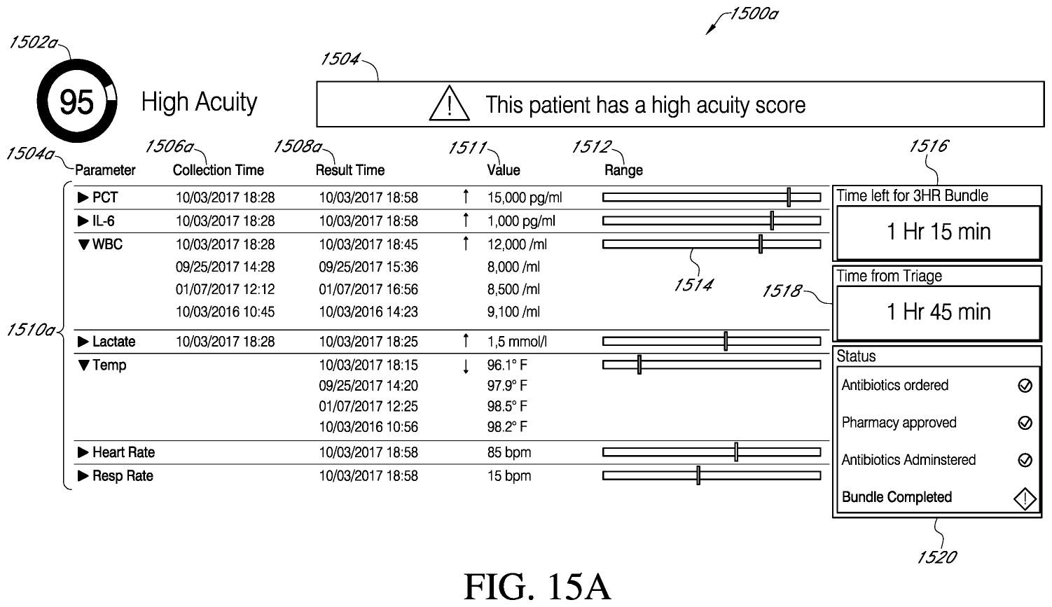

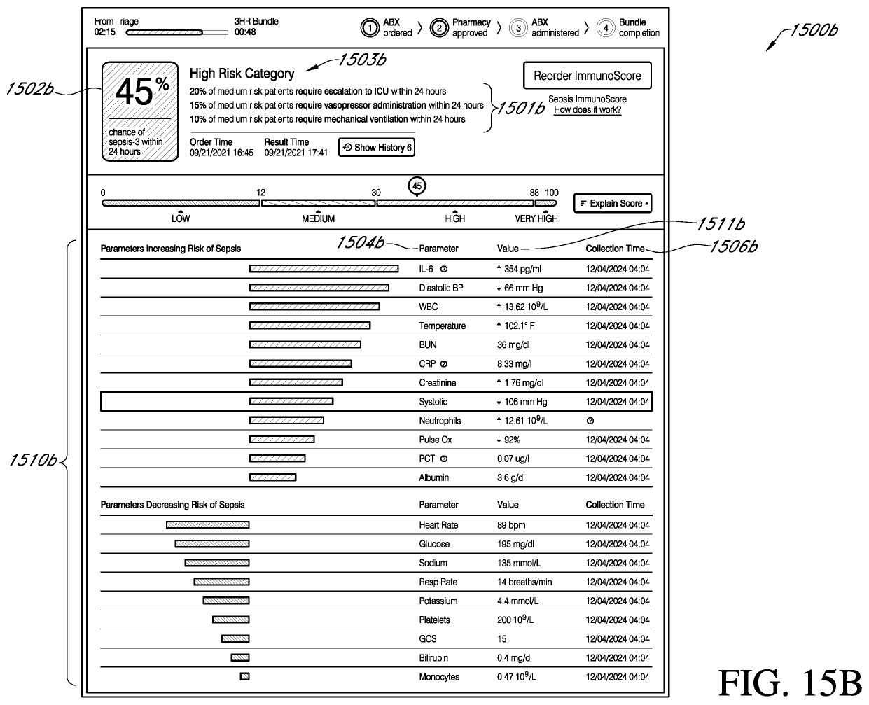

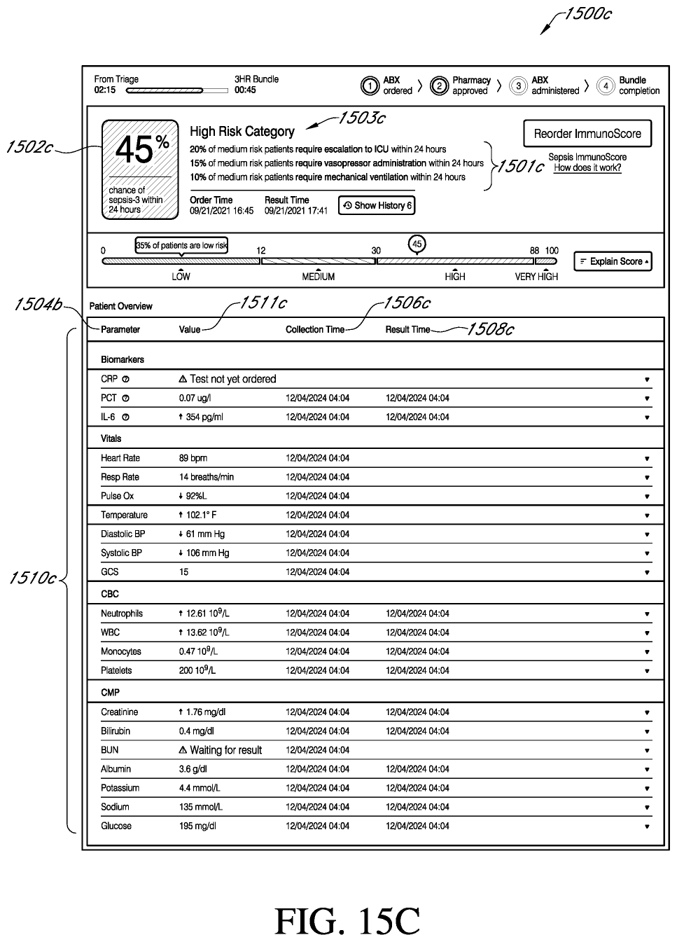

FIG. 15A illustrates an exemplary dashboard GUI 1500a for dysregulated host response monitoring including the parameter information seen in FIG. 14B and an upper panel including additional information (e.g., acuity score, acuity indicator, prognostic value, and prognostic indicator), in accordance with disclosed embodiments. FIG. 15B illustrates another exemplary dashboard GUI 1500b for dysregulated host response monitoring, in accordance with various embodiments. FIG. 15C illustrates an exemplary dashboard GUI 1500c for dysregulated host response monitoring, in accordance with various embodiments. In various embodiments, the exemplary dashboard in FIGS. 15A, 15B, and 15C may include a score panel or score dashboard GUI, in accordance with various embodiments. In some embodiments, dashboard GUI (see e.g., 1500a, 1500b, 1500c, or the like) may be displayed by client devices 110. In other embodiments, dashboard GUI (see e.g., 1500a, 1500b, 1500c, or the like) may be generated by servers 130 and transmitted through network 150 for display. For example, in some embodiments dashboard GUI (see e.g., 1500a, 1500b, 1500c, or the like) may be configured and transmitted through APIs in client devices 110 or servers 130. The dashboard GUI (see e.g., 1500a, 1500b, 1500c, or the like) may include an acuity score indicator (see e.g., 1502a, 1502b, 1502c, or the like) showing an acuity score (e.g., 45% shown in FIG. 15B). In various embodiments, the score is an output of a machine learning model. In various embodiments, the dashboard GUI may display an acuity indicator for the acuity score 1503b, 1503c.

In various embodiments, the dashboard GUI (see e.g., 1500b, 1500c or the like) may include a list 1501b, 1501c one or more risk categories (e.g., an ICU treatment category, a vasopressor administration category, a mechanical ventilation category, or possible treatments for a patient) associated with a prognostic value (e.g., a probability of an adverse event occurring—5%, 10%, etc.). In various embodiments, each risk category of one or more prognostic values may be indicated in the list 1501b, 1501c by a line including a percentage and text. In various embodiments, the text can include a percentage (e.g., a prognostic value) and a description of the adverse event. In various embodiments, the dashboard GUI (see e.g., 1500b, 1500c or the like) may display a treatment or action for each risk category of the prognostic value(s). In various embodiments, the dashboard GUI (see e.g., 1500b, 1500c or the like) may display a likely outcome for each risk category of the prognostic values. In various embodiments, the dashboard GUI (see e.g., 1500b, 1500c or the like) may display a likely of a patient having sepsis (e.g., an acuity score 1502a, 1502b, 1502c). In various embodiments, a risk category may be determined by the prognostic value or an acuity score (e.g., In various embodiments, the machine learning model may select a risk category associated with the acuity score or prognostic value. In various embodiments, the dashboard GUI (see e.g., 1500b, 1500c or the like) may display a summary of this output and potential recommended interventions (e.g., escalation to ICU, vasopressor administration, renal replacement therapy, extended length of stay, or mechanical ventilation). A parameter summary box (see e.g., 1510a, 1510b, 1510c, or the like) may display one or more timer boxes (e.g., 1516, 1518, or the like), and/or a treatment workflow box (e.g., see 1520, or the like). The dashboard GUI (see e.g., 1500b, 1500c or the like) may also include a graphical representation of the risk categories on a horizontal bar, sized according to the frequency than patients fall into that category in a reference dataset. The dashboard GUI (see e.g., 1500b, 1500c or the like) may display prognostic values associated with the risk group, representing chance of adverse events for an average patient in that risk group, in accordance with various embodiments. The parameter summary box (see e.g., 1510a, 1510b, 1510c, or the like) may include many parameter lines, potentially displayed in order using an influence score ranking according to method 1400, potentially displayed with accompanied graphical representation of the individual parameter influence scores. Parameter summary box (see e.g., 1510a, 1510b, 1510c, or the like) may also include a parameter indicator (see e.g., 1504a, 1504b, 1504c, or the like). The collection time indicator (see e.g., 1506a, 1506b, 1506c, or the like) may display a time of collection for a sample. In various embodiments, a time of result indicator (see e.g., 1508a, 1508c, or the like) for the displayed parameters. In various embodiments, a value indicator (see e.g., 1504a, 1504b, 1504c, or the like) may display a value of a parameter. In some embodiments, a color or two-dimensional histogram 1512 may a range of values. In some embodiments, the histogram 1512 may provide a summary of the distribution of the parameters for past patients from a target population as well as where the parameter value for the specific patient lies in that distribution, in accordance with some embodiments. The timer boxes (see e.g., 1516, 1518, or the like) include timers that may indicate, to a healthcare professional, how much time is left before a regulatory or reimbursement rule is violated and/or the time that has passed from an important reference time. Finally, the status of a desirable intervention or medication can be displayed in a series of lines and status symbols for the treatment workflow box (see e.g., 1520).

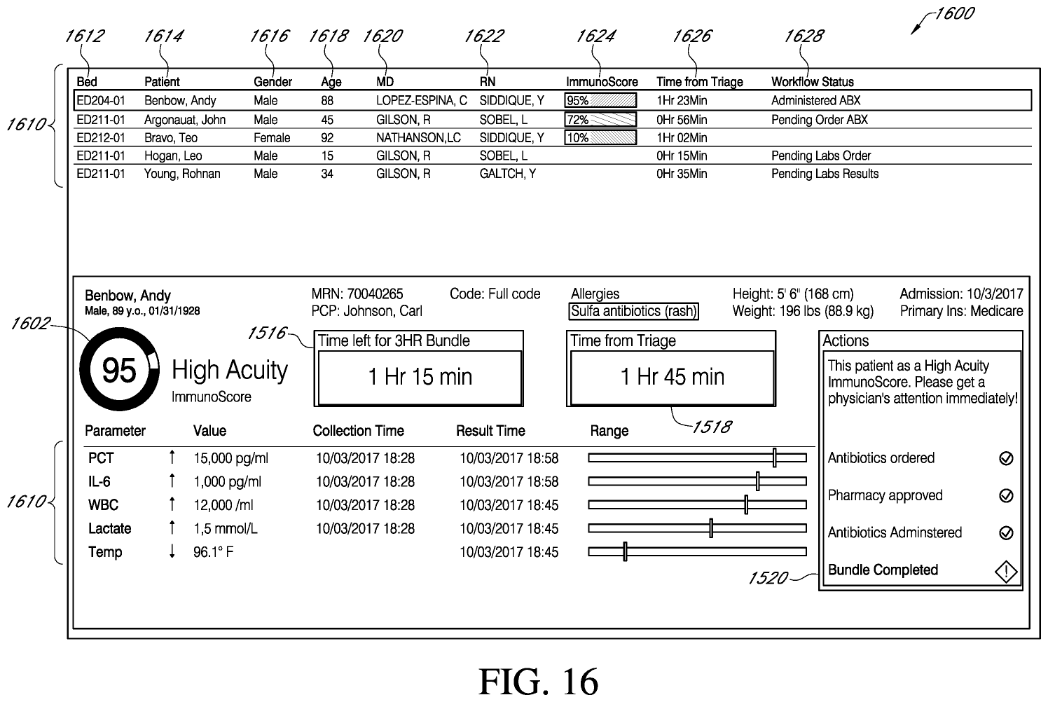

FIG. 16 illustrates a second exemplary dashboard GUI 1600 for dysregulated host response monitoring, in accordance with various embodiments. In some embodiments, dashboard GUI 1600 may be displayed by client devices 110. In other embodiments, dashboard GUI 1600 may be generated by servers 130 and transmitted through network 150 for display. For example, in some embodiments dashboard GUI 1600 may be configured and transmitted through APIs in client devices 110 or servers 130. The dashboard GUI 1600 may include elements of dashboard GUI (see e.g., 1500a, 1500b, 1500c, or the like) or any of the other dashboards described herein. In various embodiments, the dashboard GUI 1600 may display summary box 1610 of patients of interest. The summary box 1610 may display a pharmacist, sepsis coordinator, or other healthcare professional associated with each patient of interest. The elements of dashboard GUI (see e.g., 1500a, 1500b, 1500c, or the like) may be displayed for each of the patients listed in the summary box 1610. The summary box 1610 may display information relating to the location of the patient 1612, the patient's name 1614, the patient's gender 1616, the patient's age 1618, the primary physician in charge of the patient 1620, the primary nurse in charge of the patient 1622, an acuity score 1602 of an output of the machine learning model. In various embodiments, a market may comprise one or more colors, shading, text, highlighting, or some other known method of indication that may be presented on a dashboard GUI 1600. Time 1626 from an importance reference point (e.g., a collection time, a result time, time or surgery, or the like), in accordance with various embodiments. The dashboard GUI 1600 may display a workflow status 1628 of desirable treatment. The summary box 1610 may allow a healthcare professional to track patients of interest and rapidly prioritize the patients with highest risk of deterioration, in accordance with various embodiments.

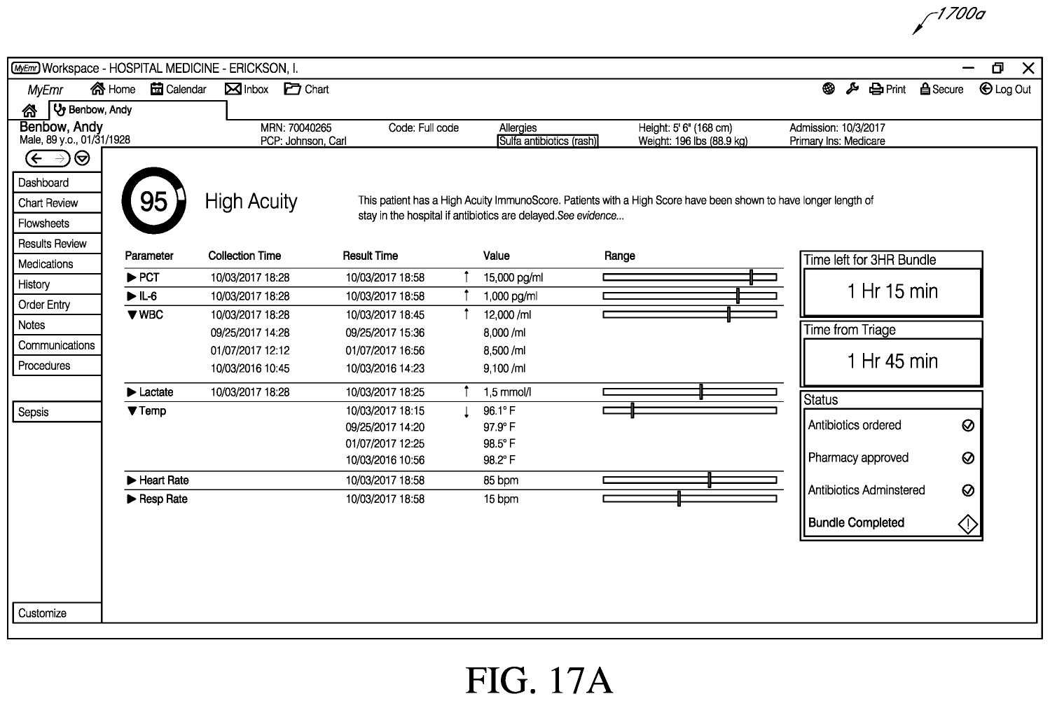

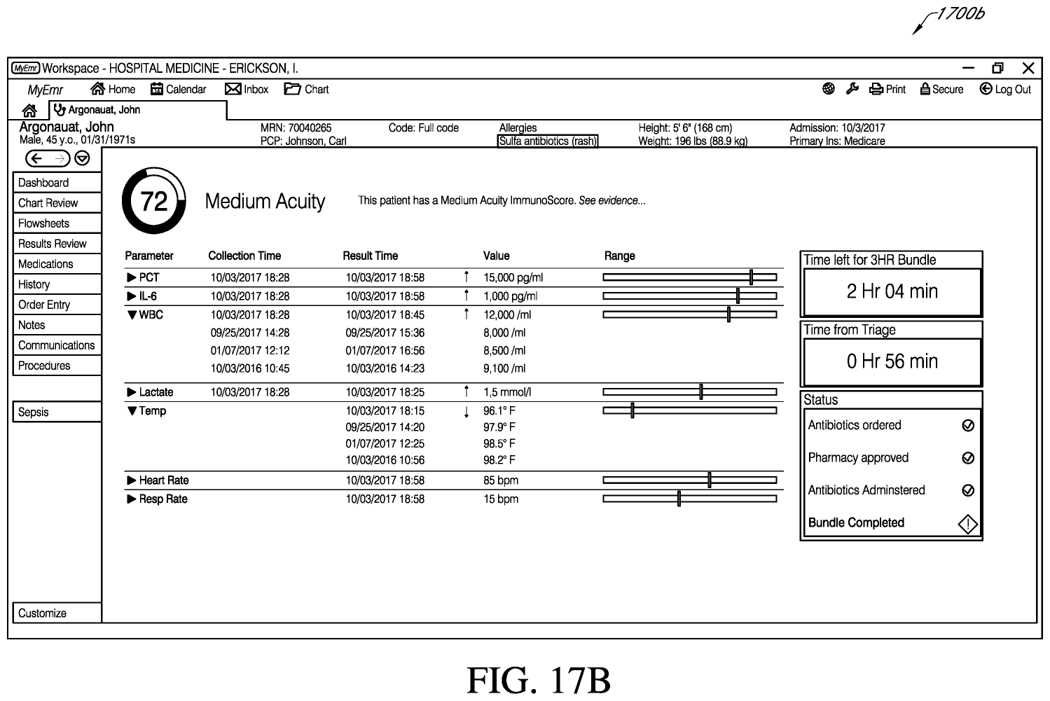

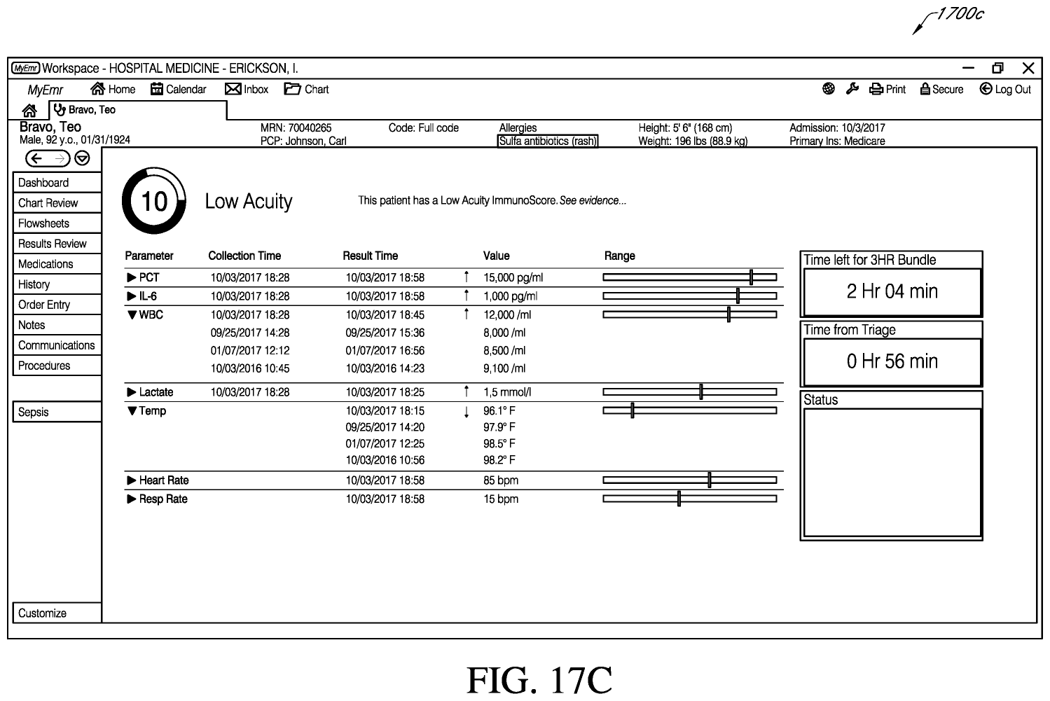

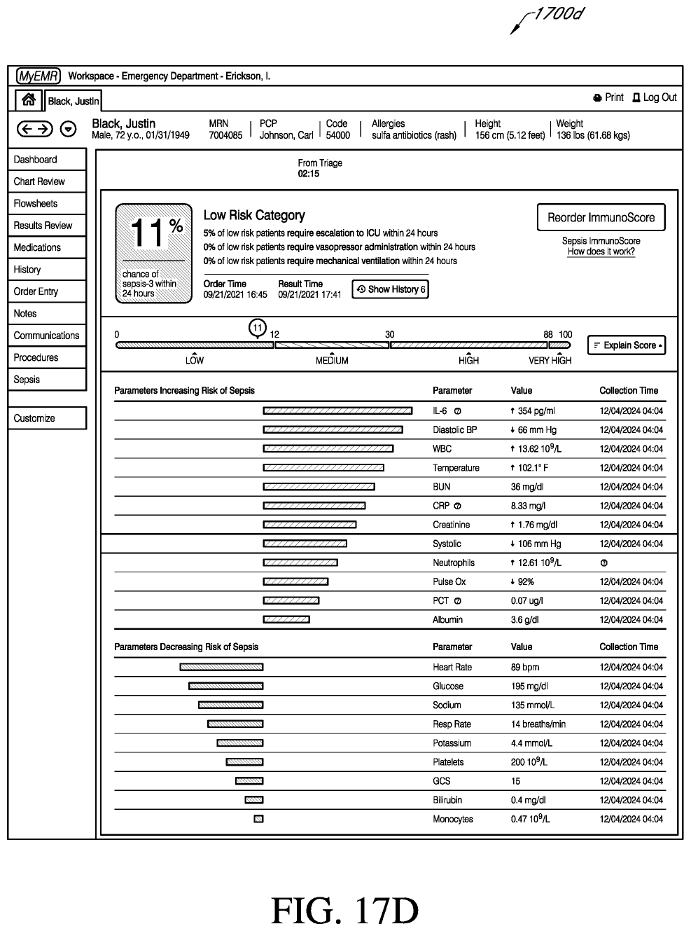

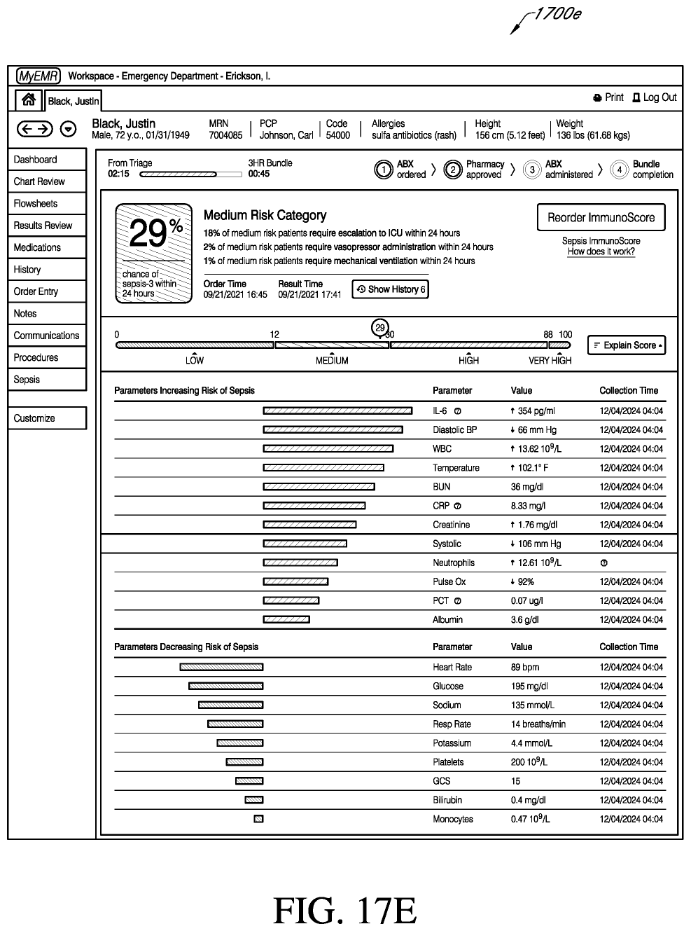

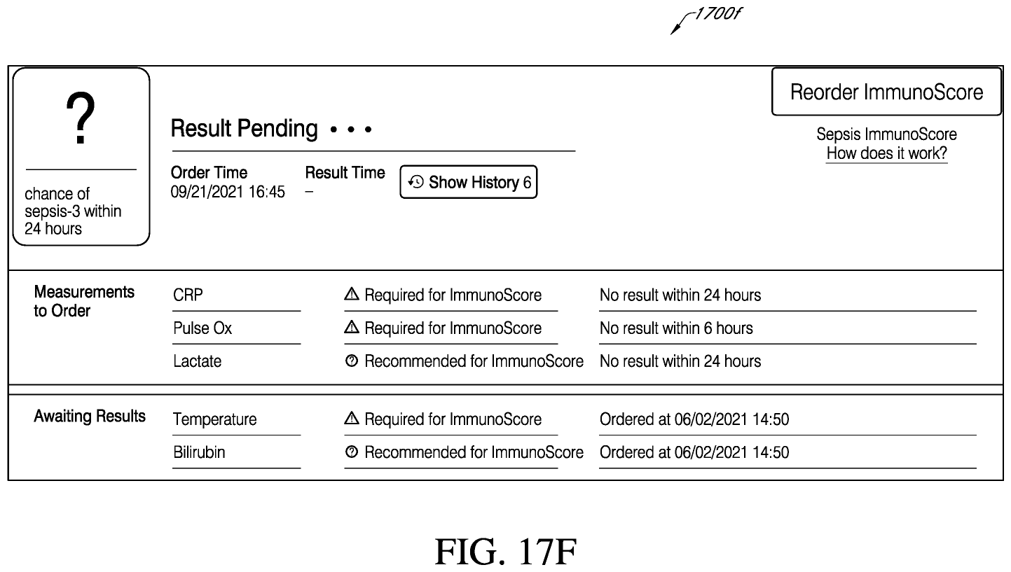

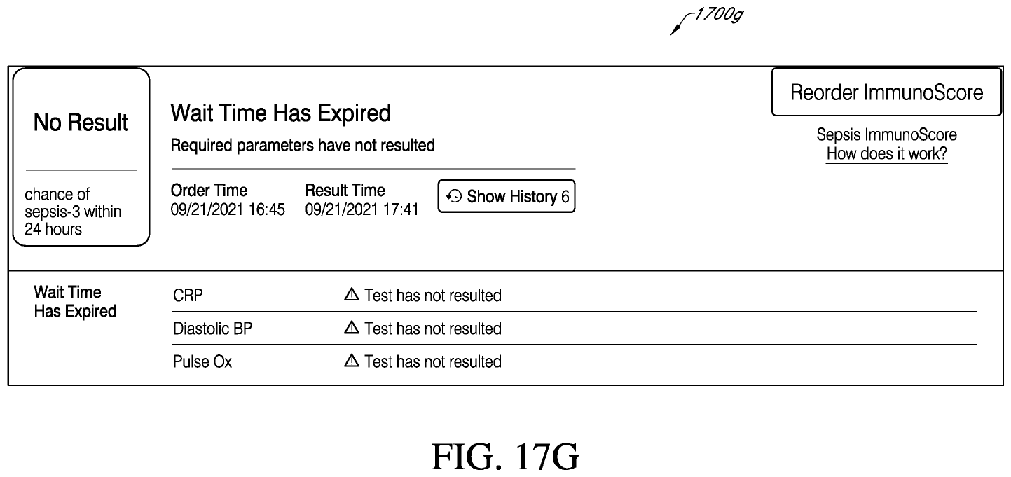

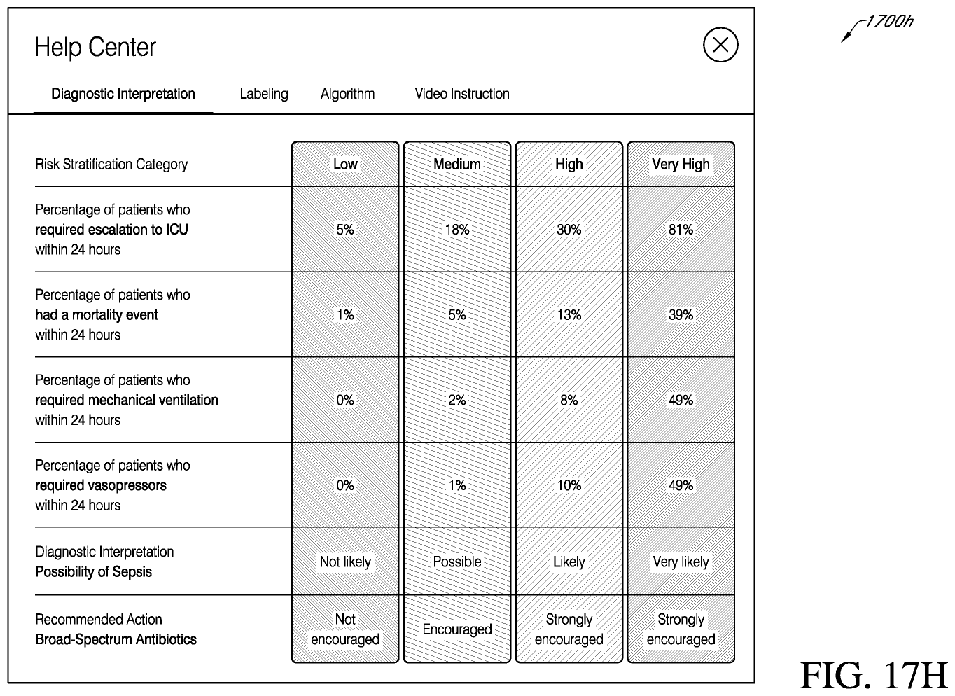

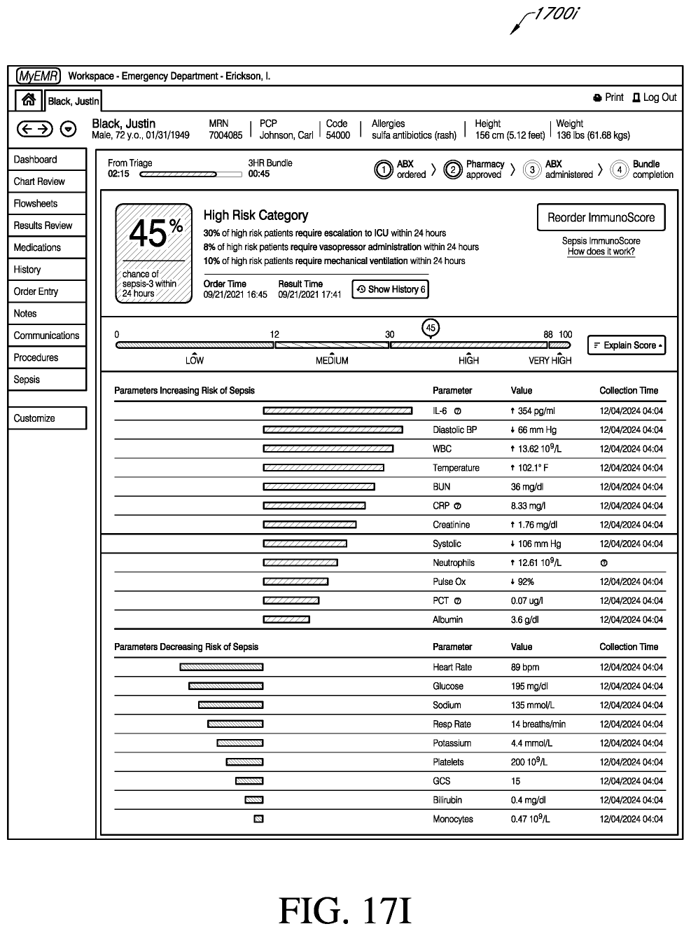

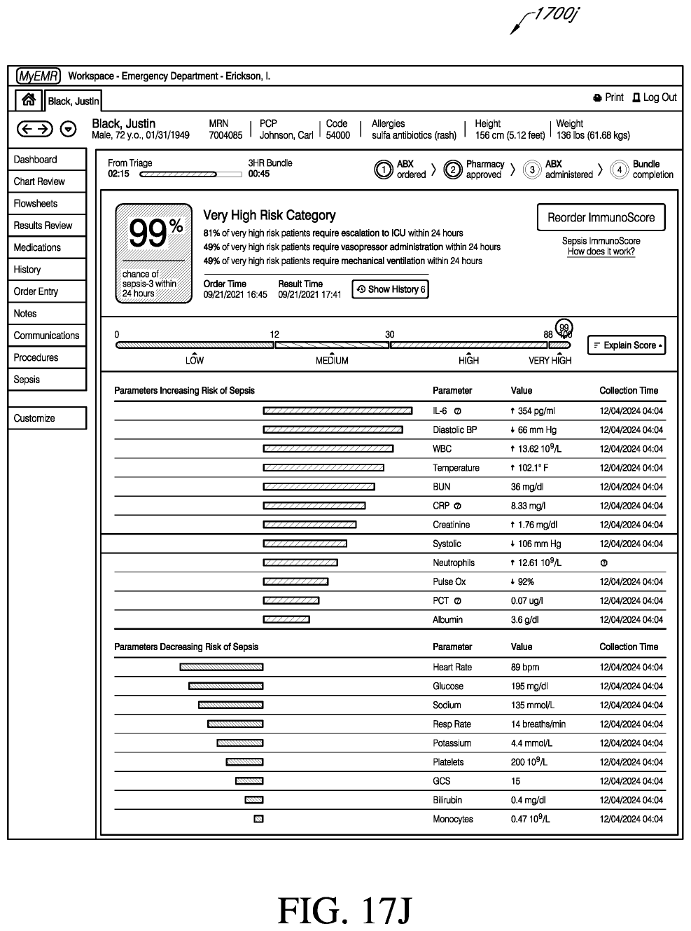

FIGS. 17A, 17B, 17C, 17D, 17E, 17F, 17G, 17H, 17I, and 17J each illustrate an exemplary dashboard GUI (see e.g., 1700a, 1700b, 1700c, 1700d, 1700e, 1700f, 1700g, 1700h, 1700i, 1700j, or the like), or portion thereof, in accordance with various embodiments.

FIG. 17A illustrates an exemplary dashboard GUI 1700a presenting a high acuity score in a browser interface, according to various embodiments.

FIG. 17B illustrates an exemplary dashboard GUI 1700b presenting a medium acuity score in a browser interface, in accordance with various embodiments.

FIG. 17C illustrates an exemplary dashboard GUI 1700c presenting a low acuity score in a browser interface, in accordance with various embodiments.

FIG. 17D illustrates an exemplary dashboard GUI 1700d presenting an acuity score associated with a low-risk acuity indicator as well three risk categories showing prognostic values, in accordance with various embodiments.

FIG. 17E illustrates an exemplary dashboard GUI 1700e presenting an acuity score associated with a medium-risk acuity indicator as well three risk categories showing prognostic values, in accordance with various embodiments.

FIG. 17F illustrates an exemplary dashboard GUI 1700f presenting a results pending indicator, in accordance with various embodiments. In various embodiments, the algorithm may not have enough information to generate an acuity score. In such embodiments, an indicator showing that the result is still pending may be displayed on the dashboard GUI 1700f.

FIG. 17G illustrates an exemplary dashboard GUI 1700g presenting a results no indicator, in accordance with various embodiments. In various embodiments, the algorithm may not have enough information to generate an acuity score. In such embodiments, an indicator showing that there is no result may be displayed on the dashboard GUI 1700f.

FIG. 17I illustrates an exemplary dashboard GUI 1700i presenting an acuity score associated with a high-risk acuity indicator as well three risk categories showing prognostic values, in accordance with various embodiments.

FIG. 17J illustrates an exemplary dashboard GUI 1700j presenting an acuity score associated with a very high-risk acuity indicator as well three risk categories showing prognostic values, in accordance with various embodiments. In some cases, an acuity score may correlate to one or more prognostic values (e.g., when one or more prognostic values are high an acuity score is also high).

In various embodiments, the dashboard GUI (see e.g., 1700a, 1700b, 1700c, 1700d, 1700e, 1700f, 1700g, 1700h, 1700i, 1700j, or the like) for patients with having one or more different risk categories of dysregulated host response for monitoring on user devices may be displayed. In some embodiments, dashboard GUI (see e.g., 1700a, 1700b, 1700c, 1700d, 1700e, 1700f, 1700g, 1700h, 1700i, 1700j, or the like) may be displayed on client devices 110. In other embodiments, dashboard GUI (see e.g., 1700a, 1700b, 1700c, 1700d, 1700e, 1700f, 1700g, 1700h, 1700i, 1700j, or the like) may be generated by servers 130 and transmitted through network 150 for display. For example, in some embodiments dashboard GUI (see e.g., 1700a, 1700b, 1700c, 1700d, 1700e, 1700f, 1700g, 1700h, 1700i, 1700j, or the like) may be configured and transmitted through APIs in client devices 110 or servers 130. Dashboard GUI (see e.g., 1700a, 1700b, 1700c, 1700d, 1700e, 1700f, 1700g, 1700h, 1700i, 1700j, or the like) may display an indicator for a patient with a high-risk score (e.g., a percentage chance of having or developing sepsis), in accordance with various embodiments. Dashboard GUI 1700b may display an indicator for a patient with a medium acuity risk score (e.g., a percentage chance of contracting sepsis). In various embodiments, dashboard GUI 1700c displays a display indicator for a patient with a low acuity risk score (e.g., a percentage chance of contracting sepsis), in accordance with various embodiments. In various embodiments, dashboard GUI 1700d displays an indicator for a patient with a low-risk category, in accordance with various embodiments. In various embodiments, dashboard GUI 1700e displays an indicator for a patient in a medium risk category, in accordance with various embodiments. In various embodiments, dashboard GUI 1700i displays an indicator for a patient in a medium risk category, in accordance with various embodiments. In various embodiments, dashboard GUI 1700f displays an indicator for a patient in a medium risk category, in accordance with various embodiments. In the case of the low acuity risk score (e.g., a percentage chance of contracting sepsis), dashboard GUI 1700a may not display a treatment workflow box because treatment would likely not be recommended for this patient at this point in time.

In various embodiments, a dashboard GUI (see e.g., 1700f, 1700g, 1700h, 1700i, 1700j, or the like) may display an indicator displaying a result status, in accordance with various embodiments. In various embodiments, the indicator displaying the result status, may inform a healthcare profession that a result is pending. In various embodiments, the indicator displaying the result status, may inform a healthcare profession that there is no result. In various embodiments, the machine learning algorithm may determine that not enough relevant data has been input to apply the algorithm.

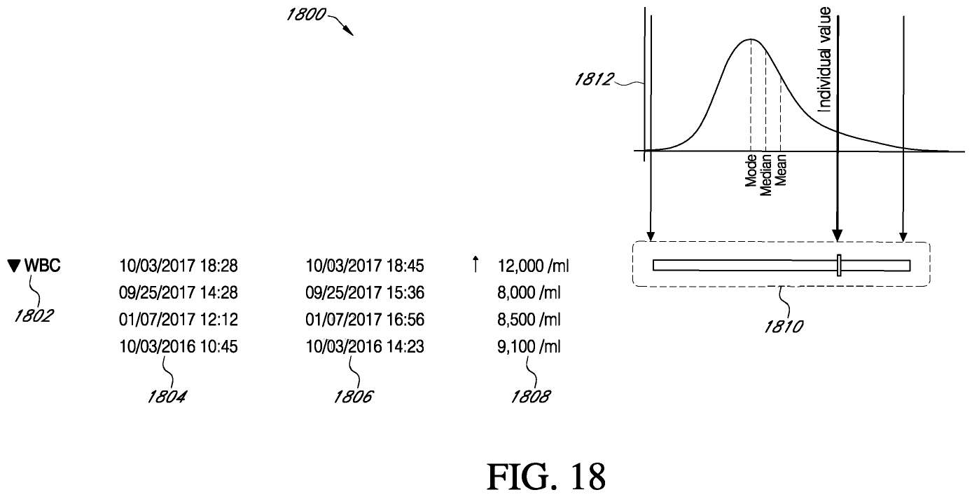

FIG. 18 illustrates an exemplary dashboard GUI 1800, or portion thereof, for identifying and displaying the value of parameters with respect to a reference population selected by user, according to various embodiments. In some embodiments, dashboard GUI 1800 may be displayed by client devices 110. In other embodiments, dashboard GUI 1800 may be generated by servers 130 and transmitted through network 150 for display. For example, in some embodiments dashboard GUI 1800 may get configured and transmitted through APIs in client devices 110 or servers 130.

A histogram 1812 can be generated for a single parameter or the output of a multidimensional statistical calculations performed from more than one parameter for more than one past patients from a target population. The parameter value observed for a patient at a point in time can be plotted on this histogram to give a user a visual representation of an abnormal or dysregulated host response of a patient in order of parameters of interest. In various embodiments, the parameter may be sorted by time or by any individual parameter value. The histogram can also be converted from a two-dimensional plot to a color histogram 1810, including a vertical bar indicating the value of the current patient's parameter at a point in time, in accordance with various embodiments. The histogram 1812 or range bar 1810 can be incorporated in a parameter summary box (see e.g., 1510a, 1510b, or the like) in the final GUI. In various embodiments, the histogram 1812 and/or the range bar 1810 may display an individual value for a parameter in comparison with a range of values from a population for the parameter. For example, the plot or range may include a target population distribution of a parameter (e.g., blood pressure) and the patient being monitored may have their specific value for that parameter indicated within the range or histogram to provide a visual representation of the patient's host response to host responses of the target population.

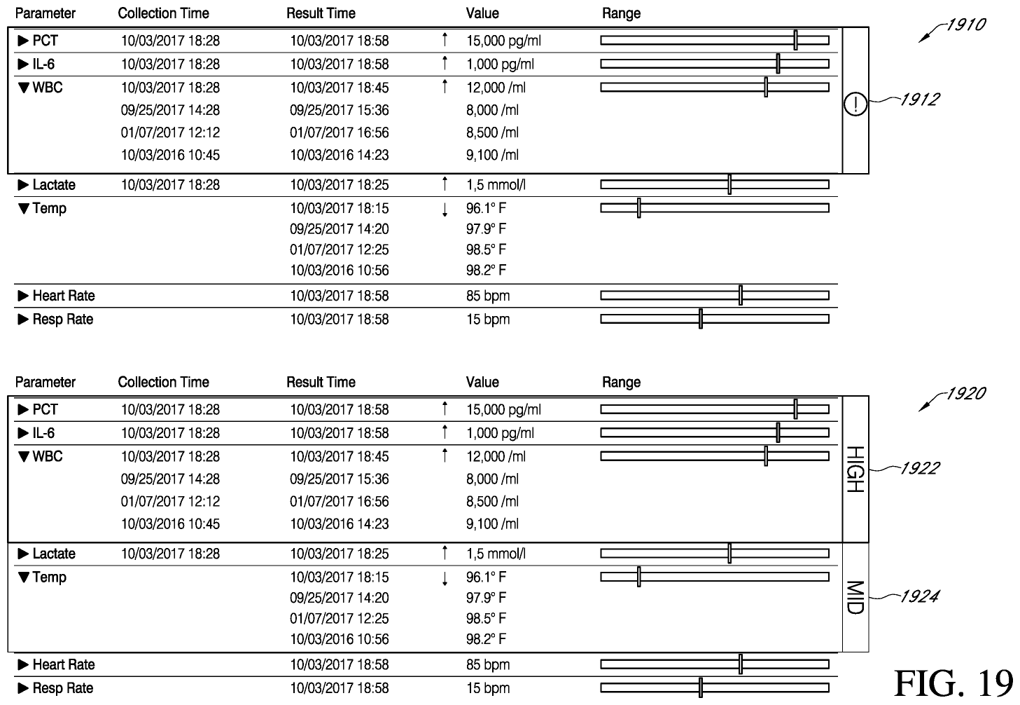

FIG. 19 illustrates exemplary dashboard GUIs (see e.g., 1910, 1920, or the like) for displaying local or global parameter importance ranking used by a machine learning model, according to various embodiments. In some embodiments, dashboard GUI (see e.g., 1910, 1920, or the like) may be displayed on client devices 110. In other embodiments, dashboard GUI (see e.g., 1900a, 1900b, or the like) may be generated by servers 130 and transmitted through network 150 for display. For example, in some embodiments dashboard GUI (see e.g.,1900a, 1900b, or the like) may be configured and transmitted through APIs in client devices 110 or servers 130.

FIG. 19A illustrates two example dashboard GUIs (see e.g., 1910, 1920, or the like), in accordance with various embodiment. Dashboard GUI 1910 is shown, where an exclamation mark is placed in a panel 1912 to highlight parameters determined to have high influence scores, in accordance with some embodiments. Second, GUI 1920 illustrates that certain parameters can be highlighted as having high, medium, or low influence scores with side panels 1922 and 1924. The parameters may be organized into risk categories which may include one or more indicators showing “increase risk” and “decrease risk”, ranked according to their influence score, with influence scores shown graphically in a bar plot.

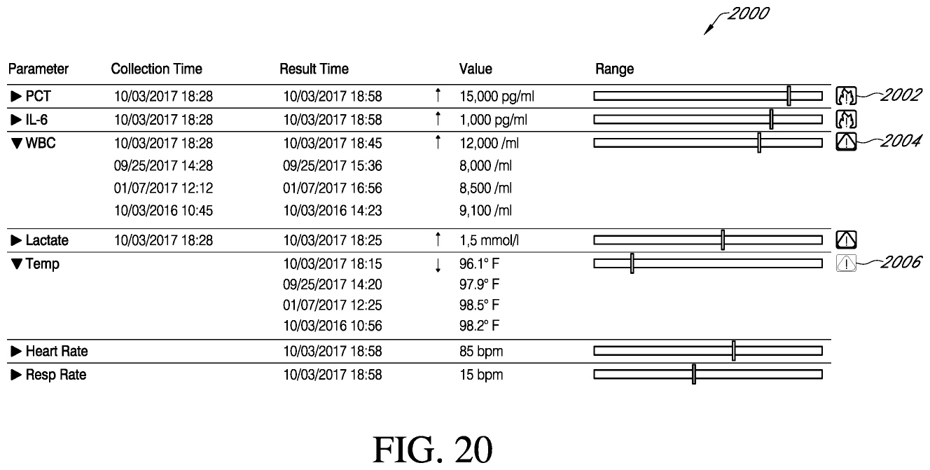

Like FIG. 19, FIG. 20 illustrates an exemplary dashboard GUI 2000 for displaying local or global parameter importance contribution used by a machine learning model, according to various embodiments. In some embodiments, dashboard GUI 2000 may be displayed by client devices 110. In other embodiments, dashboard GUI 2000 may be generated by servers 130 and transmitted through network 150 for display. For example, in some embodiments, dashboard GUI 2000 may be configured and transmitted through APIs in client devices 110 or servers 130.

In various embodiments, a description of an influence score for each parameter can be displayed using alternate symbols of very high influence score with a symbol, high influence score with a symbol, or medium influence score with a symbol. These symbols can be used in (e.g., A parameter summary box may comprise one or more of the dashboard GUIs described herein, in accordance with various embodiments.).

In various embodiments, the dashboard GUI 2000 may include one or more emphasis indicators 2002, 2004, 2006. In various embodiments, emphasis indicators can indicate the importance of a parameter for a patient.

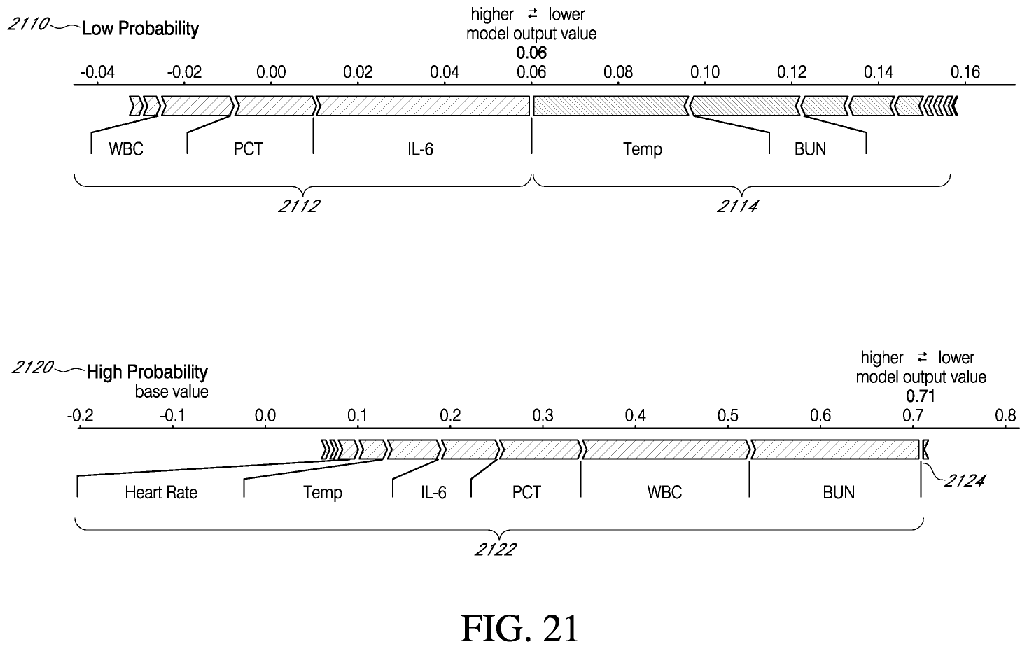

FIG. 21 illustrates exemplary dashboard GUIs (see e.g., 2110, 2120, or the like) for displaying local or global parameter importance contribution used by a machine learning model, according to various embodiments. In some embodiments, dashboard GUI (see e.g., 2110, 2120, or the like) may be displayed by client devices 110. In other embodiments, dashboard GUI (see e.g., 2110, 2120, or the like) may be generated by servers 130 and transmitted through network 150 for display. For example, in some embodiments, dashboard GUI (see e.g., 2110, 2120, or the like) may be configured and transmitted through APIs in client devices 110 or servers 130.

Two examples of Shapley Additive Explanations are illustrated (e.g. 2110 and 2120), in accordance with various embodiments. Dashboard GUI 2110 displays a diagram for a patient with a low acuity score or a low prognostic value and a diagram for a patient, in accordance with various embodiments. Dashboard GUI 2120 displays a diagram for a patient with a high acuity score or a high prognostic value. In both diagrams, the red bars 2112 and 2122 indicate parameters that have elevated the final acuity or prognostic scores. The width of each bar corresponds to the overall contribution of the particular parameter to increasing the final acuity score, in accordance with various embodiments. The blue bars 2114 and 2124 indicate parameters that have decreased the final acuity score, according to various embodiments. Once again, the width of the bars corresponds to the overall contribution of the particular parameter to decreasing the final acuity score. These diagrams may also be displayed in a parameter summary box on the final dashboard GUI to increase interpretability, intuition, and trust in the overall machine learning model for a healthcare professional user.

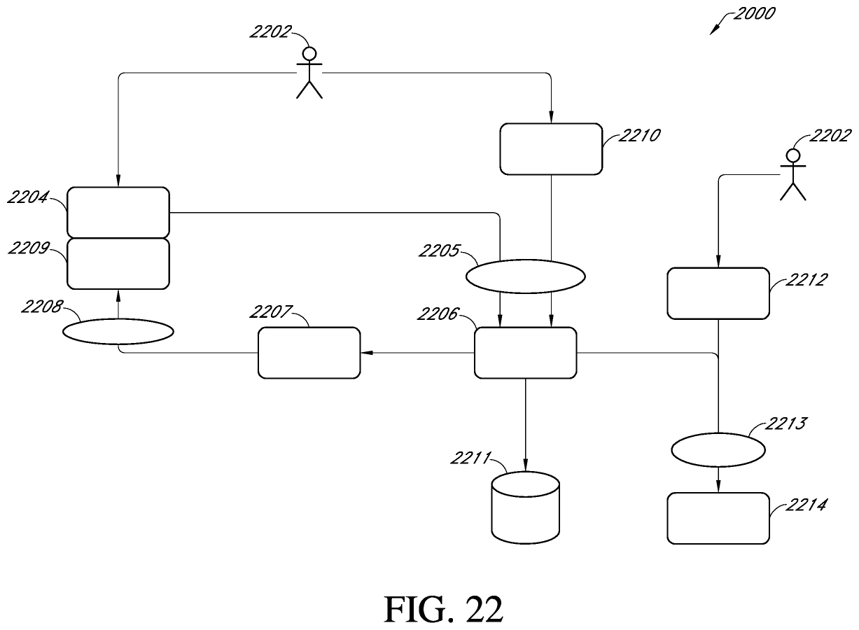

FIG. 22 is a block diagram 2200 illustrating an example software system utilizing the described systems, including the necessary components and interfacing, according to various embodiments. In accordance with some embodiments, elements of block diagram 2200 may be implemented with architecture 100 or block diagram 200. Further, at least some elements of block diagram 2200 may be performed by a computer having a processor executing commands stored in a memory of the computer.

The healthcare professional 2202 can access the software interface 2206 through the EMR web interface 2204 or, alternatively or in addition to, directly through a web browser 2210, in accordance with various embodiments. In both cases, authentication 2205 may be mediated through the OAuth 2.0 protocol with the EMR Server, or the like.

The software interface 2206 may fetch information about patients from the EMR server API 2209 mediated by SMART FHIR authentication 2208. The information may be stored in a database 2211. This information about patients may be presented to the healthcare professional 2202 when they access the software interface 2206. In various embodiments, a middleware 2207 (e.g., FHIR API Middleware) may be used to facilitate transfer of patient parameters (“parameter” used interchangeable with “feature”) to other components of the system (e.g., SMART FHIR Auth). The software interface 2206 fetches information about prognostic value, prognostic

indicator, acuity score, risk category, treatment guidance, readiness flag, parameters used as input and/or output, and the influence score for each parameter from algorithm 2214, mediated by an authentication system managed by authentication 2213. The information fetched by the software interface 2206 may be stored in the database 2211. This information may be presented to the healthcare professional 2202 when they access the software interface 2206.

Independently from the software interface 2206, a healthcare professional 2202 can access the algorithm 2214 directly through a web browser 2212, mediated with the same authentication 2213. The healthcare professional 2202 can access prognostic value, prognostic indicator, acuity score, risk category, treatment guidance, readiness flag, parameters used for input and/or output, and the influence score for each parameter in an independent manner, outside of the software interface 2206.

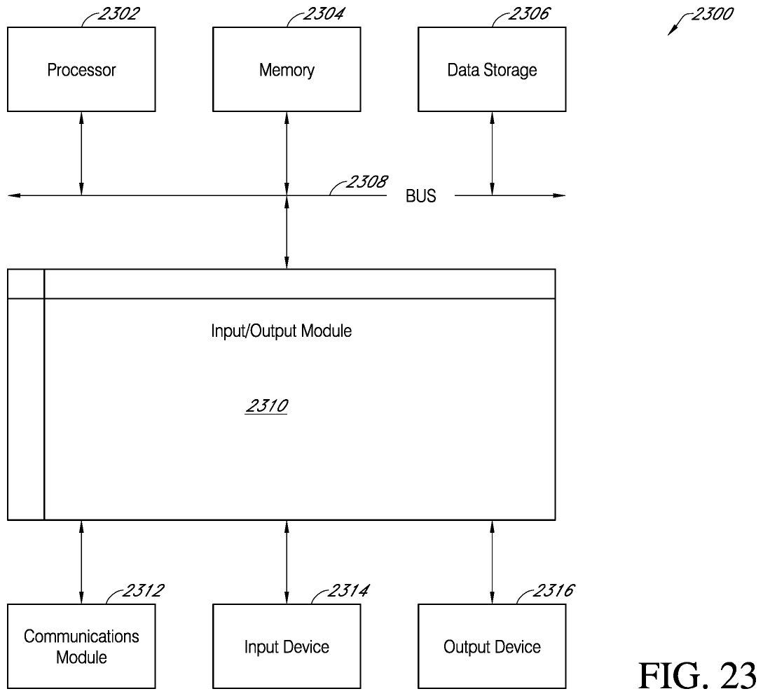

FIG. 23 is a block diagram illustrating an exemplary computer system 2300 with which the client device 110 and server 130 described herein (see e.g., FIGS. 1 and 2), and the methods and dashboard GUIs illustrated and described herein can be implemented, in accordance with various embodiments. In certain aspects, the computer system 2300 may be implemented using hardware or a combination of software and hardware, either in a dedicated server, or integrated into another entity, or distributed across multiple entities, in accordance with various embodiments.

Computer system 2300 (e.g., client device 110 and server 130) may include a bus 2308 or other communication mechanism for communicating information, and a processor 2302 (e.g., processors 212) coupled with bus 2308 for processing information. By way of example, the computer system 2300 may be implemented with one or more processors 2302. processor 2302 may be a general-purpose microprocessor, a microcontroller, a digital signal processor (DSP), an application specific integrated circuit (ASIC), a field programmable gate array (FPGA), a programmable logic device (PLD), a controller, a state machine, gated logic, discrete hardware components, or any other suitable entity that can perform calculations or other manipulations of information.

Computer system 2300 can include, in addition to hardware, code that creates an execution environment for the computer program in question, e.g., code that constitutes processor firmware, a protocol stack, a database management system, an operating system, or a combination of one or more of them stored in an included memory 2304 (e.g., memories 220), such as a random access memory (RAM), a flash memory, a read-only memory (ROM), a programmable read-only memory (PROM), an erasable PROM (EPROM), registers, a hard disk, a removable disk, a CD-ROM, a DVD, or any other suitable storage device, coupled to bus 2308 for storing information and instructions to be executed by processor 2302. The processor 2302 and the memory 2304 can be supplemented by, or incorporated in, special purpose logic circuitry.

The instructions may be stored in the memory 2304 and implemented in one or more computer program products, i.e., one or more modules of computer program instructions encoded on a computer-readable medium for execution by, or to control the operation of, the computer system 2300, and according to any method well known to those of skill in the art, including, but not limited to, computer languages such as data-oriented languages (e.g., SQL, dBase), system languages (e.g., C, Objective-C, C++, Assembly), architectural languages (e.g., Java, .NET), and application languages (e.g., PHP, Ruby, Perl, Python). Instructions may also be implemented in computer languages such as array languages, aspect-oriented languages, assembly languages, authoring languages, command line interface languages, compiled languages, concurrent languages, curly-bracket languages, dataflow languages, data-structured languages, declarative languages, esoteric languages, extension languages, fourth-generation languages, functional languages, interactive mode languages, interpreted languages, iterative languages, list-based languages, little languages, logic-based languages, machine languages, macro languages, metaprogramming languages, multi paradigm languages, numerical analysis, non-English-based languages, object-oriented class-based languages, object-oriented prototype-based languages, off-side rule languages, procedural languages, reflective languages, rule-based languages, scripting languages, stack-based languages, synchronous languages, syntax handling languages, visual languages, with languages, and xml-based languages. Memory 2304 may also be used for storing temporary variables or other intermediate information during execution of instructions to be executed by processor 2302.

A computer program as discussed herein does not necessarily correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, subprograms, or portions of code). A computer program can be deployed to be executed on one computer or on multiple computers that are located at one site or distributed across multiple sites and interconnected by a communication network. The processes and logic flows described in this specification can be performed by one or more programmable processors executing one or more computer programs to perform functions by operating on input data and generating output.

Computer system 2300 further includes a data storage device 2306 such as a magnetic disk or optical disk, coupled to bus 2308 for storing information and instructions. Computer system 2300 may be coupled via input/output module 2310 to various devices. Input/output module 2310 can be any input/output module. Exemplary input/output modules 2310 include data ports such as USB ports. The input/output module 2310 is configured to connect to a communications module 2312. Exemplary communications modules 2312 (e.g., communications modules 218) include networking interface cards, such as Ethernet cards and modems. In certain aspects, input/output module 2310 is configured to connect to a plurality of devices, such as an input device 2314 (e.g., input device 214) and/or an output device 2316 (e.g., output device 216). Exemplary input devices 2314 include a keyboard and a pointing device, e.g., a mouse or a trackball, by which a user can provide input to the computer system 2300. Other kinds of input devices 2314 can be used to provide for interaction with a user as well, such as a tactile input device, visual input device, audio input device, or brain-computer interface device. For example, feedback provided to the user can be any form of sensory feedback, e.g., visual feedback, auditory feedback, or tactile feedback; and input from the user can be received in any form, including acoustic, speech, tactile, or brain wave input. Exemplary output devices 2316 include display devices, such as an LCD (liquid crystal display) monitor, for displaying information to the user.

According to one aspect of the present disclosure, the client device 110 and server 130 can be implemented using a computer system 2300 in response to processor 2302 executing one or more sequences of one or more instructions contained in memory 2304. Such instructions may be read into memory 2304 from another machine-readable medium, such as data storage device 2306. Execution of the sequences of instructions contained in main memory 2304 causes processor 2302 to perform the process steps described herein. One or more processors in a multi-processing arrangement may also be employed to execute the sequences of instructions contained in memory 1404. In various aspects, hard-wired circuitry may be used in place of or in combination with software instructions to implement various aspects of the present disclosure. Thus, aspects of the present disclosure are not limited to any specific combination of hardware circuitry and software.

Various aspects of the subject matter described in this specification can be implemented in a computing system that includes a back end component, e.g., as a data server, or that includes a middleware component, e.g., an application server, or that includes a front end component, e.g., a client computer having a graphical user interface or a Web browser through which a user can interact with an implementation of the subject matter described in this specification, or any combination of one or more such back end, middleware, or front end components. The components of the system can be interconnected by any form or medium of digital data communication, e.g., a communication network. The communication network (e.g., network 150) can include, for example, any one or more of a LAN, a WAN, the Internet, and the like. Further, the communication network can include, but is not limited to, for example, any one or more of the following network topologies, including a bus network, a star network, a ring network, a mesh network, a star-bus network, tree or hierarchical network, or the like. The communications modules can be, for example, modems or Ethernet cards.

Computer system 2300 can include clients and servers. A client and server are generally remote from each other and typically interact through a communication network. The relationship of client and server arises by virtue of computer programs running on the respective computers and having a client-server relationship to each other. Computer system 2300 can be, for example, and without limitation, a desktop computer, laptop computer, or tablet computer. Computer system 2300 can also be embedded in another device, for example, and without limitation, a mobile telephone, a PDA, a mobile audio player, a Global Positioning System (GPS) receiver, a video game console, and/or a television set top box.

The term “machine-readable storage medium” or “computer-readable medium” as used herein refers to any medium or media that participates in providing instructions to processor 2302 for execution. Such a medium may take many forms, including, but not limited to, non-volatile media, volatile media, and transmission media. Non-volatile media include, for example, optical or magnetic disks, such as data storage device 2306. Volatile media include dynamic memory, such as memory 23204. Transmission media include coaxial cables, copper wire, and fiber optics, including the wires that include bus 2308. Common forms of machine-readable media include, for example, floppy disk, a flexible disk, hard disk, magnetic tape, any other magnetic medium, a CD-ROM, DVD, any other optical medium, punch cards, paper tape, any other physical medium with patterns of holes, a RAM, a PROM, an EPROM, a FLASH EPROM, any other memory chip or cartridge, or any other medium from which a computer can read. The machine-readable storage medium can be a machine-readable storage device, a machine-readable storage substrate, a memory device, a composition of matter affecting a machine-readable propagated signal, or a combination of one or more of them.

As used herein, the phrase “at least one of” preceding a series of items, with the terms “and” or “or” to separate any of the items, modifies the list as a whole, rather than each member of the list (i.e., each item). The phrase “at least one of” does not require selection of at least one item; rather, the phrase allows a meaning that includes at least one of any one of the items, and/or at least one of any combination of the items, and/or at least one of each of the items. By way of example, the phrases “at least one of A, B, and C” or “at least one of A, B, or C” each refer to only A, only B, or only C; any combination of A, B, and C; and/or at least one of each of A, B, and C. To the extent that the term “include,” “have,” or the like is used in the description or the claims, such term is intended to be inclusive in a manner similar to the term “comprise” as “comprise” is interpreted when employed as a transitional word in a claim. The word “exemplary” is used herein to mean “serving as an example, instance, or illustration.” Any embodiment described herein as “exemplary” is not necessarily to be construed as preferred or advantageous over other embodiments.

A reference to an element in the singular is not intended to mean “one and only one” unless specifically stated, but rather “one or more.” All structural and functional equivalents to the elements of the various configurations described throughout this disclosure that are known or later come to be known to those of ordinary skill in the art are expressly incorporated herein by reference and intended to be encompassed by the subject technology. Moreover, nothing disclosed herein is intended to be dedicated to the public regardless of whether such disclosure is explicitly recited in the above description.

While this specification contains many specifics, these should not be construed as limitations on the scope of what may be claimed, but rather as descriptions of particular implementations of the subject matter. Certain parameters that are described in this specification in the context of separate embodiments can also be implemented in combination in a single embodiment. Conversely, various parameters that are described in the context of a single embodiment can also be implemented in multiple embodiments separately or in any suitable sub-combination. Moreover, although parameters may be described above as acting in certain combinations and even initially claimed as such, one or more parameters from a claimed combination can in some cases be excised from the combination, and the claimed combination may be directed to a sub-combination or variation of a sub-combination.

The subject matter of this specification has been described in terms of particular aspects, but other aspects can be implemented and are within the scope of the following claims. For example, while operations are depicted in the drawings in a particular order, this should not be understood as requiring that such operations be performed in the particular order shown or in sequential order, or that all illustrated operations be performed, to achieve desirable results. The actions recited in the claims can be performed in a different order and still achieve desirable results. As one example, the processes depicted in the accompanying figures do not necessarily require the particular order shown, or sequential order, to achieve desirable results. In certain circumstances, multitasking and parallel processing may be advantageous. Moreover, the separation of various system components in the aspects described above should not be understood as requiring such separation in all aspects, and it should be understood that the described program components and systems can generally be integrated together in a single software product or packaged into multiple software products. Other variations are within the scope of the following claims.

### Recitation of Embodiments

Embodiment 1. A system for generating an interactive dashboard graphical user interface (GUI) for displaying indicators based on one or more acuity values, associated with a risk category, the system comprising one or more processors and one or more memory devices, wherein the one or more memory devices comprise instructions that, when executed by the processor, configure the one or more processors to perform operations comprising transmitting a patient ID to a management platform, receiving, from the management platform, at least one electronic record associated with a patient, the at least one electronic record comprising patient data, employing a machine learning model to generate an acuity score based on the patient data, the acuity score representing probability and level of a type of host response by the patient, employing a machine learning model to generate one or more prognostic values based on the patient data, the prognostic value representing a probability of an adverse event, determining a ranking of the at least one parameter according to an influence score associated with the at least one parameter, and generating a dashboard GUI for displaying, on or more client devices, the dashboard GUI comprising an acuity indicator displaying the acuity score and an associated risk category, at least one prognostic indicator displaying the at least one prognostic value and one or more risk categories associated with the at least one prognostic value, and a list displaying the parameters according to the ranking.

Embodiment 2. The system of Embodiment 1, wherein the acuity score comprises a probability of the patient currently experiencing or developing the type of host response due to stimuli within a time period.

Embodiment 3. The system of Embodiment 2, wherein the type of host response includes an undesirable host response.

Embodiment 4. The system according to any one of Embodiments 2-3, wherein the stimuli includes at least one of infection, therapy, or trauma.

Embodiment 5. The system of Embodiment 4, wherein the at least one of infection, therapy, or trauma includes sepsis.

Embodiment 6. The system of Embodiment 2, wherein the time period is 24 hours or less.

Embodiment 7. The system according to any one of the preceding Embodiments, wherein the adverse events comprise at least one of death, 30-day readmission, escalation to ICU, vasopressor administration, renal replacement therapy, extended length of stay, increased cost of patient stay, extracorporeal membrane oxygenation intervention, or mechanical ventilation.

Embodiment 8. The system according to any one of the preceding Embodiments, wherein the dashboard GUI further comprises a workflow status displaying a treatment timetable identified by the machine learning model, wherein the treatment timetable includes at least one of order time, administration time, treatment time.

Embodiment 9. The system according to any of the preceding Embodiments wherein the dashboard GUI comprises a timetable displaying selected parameters used by the machine learning model, wherein the selected parameters include at least one of a timetable displaying an order time, a blood draw time, a recorded time, and a result time.

Embodiment 10. The system according to any one of the preceding Embodiments, wherein the dashboard GUI further comprises a notification based on the prognostic value and an output from the machine learning model and one or more interactive timers, wherein the one or more interactive times comprise a first timer displaying a time from a reference point and a second timer displaying a time left to complete treatment and diagnostic actions before violating care guidelines.

Embodiment 11. The system according to any one of the preceding Embodiments, wherein the target population comprises patients suspected of having an infection.

Embodiment 12. The system of Embodiment 11, wherein the infection includes sepsis.

Embodiment 13. The system according to any one of the preceding Embodiments, wherein the dashboard GUI comprises an interactive population selector.

Embodiment 14. The system of Embodiment 13, wherein the interactive population selector comprises a scatter plot and an area selection tool configured to an area of the scatter plot.

Embodiment 15. The system according to any one of the preceding Embodiments, wherein the target population is based on pretest probability and patient location.

Embodiment 16. The system according to any one of the preceding Embodiments, wherein the target population is defined by a second machine learning model, the second machine learning model being an unsupervised model.

Embodiment 17. The system according to any one of the preceding Embodiments, wherein identifying the parameters comprises calculating a univariate distance score between the selected parameters and the parameters of the target population and determining the ranking comprises determining the ranking independently for each patient in the patient data and comparing the univariate score between the parameters.

Embodiment 18. The system according to any one of the preceding Embodiments, wherein identifying the parameters comprises calculating individual parameters contributions using at least one of SHAP (SHapley Additive exPlanations) or Mahalanobis methods and determining the ranking comprises employing the at least one of SHAP or Mahalanobis methods to compare the parameters.

Embodiment 19. The system according to any one of Embodiments 8-13, wherein the dashboard GUI further comprises an additive explanation bar plot, the selected parameters comprise at least one of patient lab results, patient biomarker results, patient clinical parameters, derivative results, or patient trajectory information, the timetable displayed on the dashboard GUI comprises interactive hide/show buttons, and the timetable displays a result time and a value for each of the selected parameters.

Embodiment 20. The system according to any one of the preceding Embodiments, wherein the dashboard GUI further comprises a checklist of treatment and diagnostic actions recommended by care guidelines for septic patients, the checklist comprising items for one or more of administration of antibiotics, ordering of blood cultures prior to antibiotics, measurement of serum lactate, administration of fluid resuscitation, or administration of vasopressors, wherein the checklist displays a status of flow for each one of the items, the status of flow specifying at least one of physician order status, pharmacy approval status, administration of medication status, or full guidelines completed status.

Embodiment 21. The system according to any one of the preceding Embodiments, wherein the dashboard GUI displayed is displayed embedded into a patient chart.

Embodiment 22. The system according to any one of the preceding Embodiments, wherein employing the machine learning model comprises storing previously collected patient data from different target populations and returning a location associated with the patient data with reference to the target population.

Embodiment 23. The system according to any one of the preceding Embodiments, wherein employing the machine learning model comprises performing an API call to a machine learning server, the API call comprising the patient data and receiving from the machine learning server the acuity score and the at least one prognostic value, the risk category of the acuity score, the one or more risk categories of the one or more prognostic values, the selected parameters, and the influence score of each parameter.

Embodiment 24. The system according to any one of the preceding Embodiments, wherein the dashboard GUI is configured to be displayed on a mobile device associated with a healthcare professional.

Embodiment 25. The system according to any one of the preceding Embodiments, wherein the operations further comprise coupling to one or more of a point of care diagnostic or a measurement device to the system and collecting a portion of the patient data directly from the point of care diagnostic or the measurement device.

Embodiment 26. The system according to any one of the preceding Embodiments, wherein the operations further comprise training the machine learning model using supervised algorithms trained using one or more labels correlating to the types of host response defined by an output of an unsupervised algorithm.

Embodiment 27. The system according to any one of the preceding Embodiments, wherein the risk category associated with the acuity score includes one of low, medium, high, or very high.

Embodiment 28. A computer implemented method for generating a dashboard GUI for displaying host response metrics, the method comprising coupling an analytics server with a management platform through a FHIR API, generating a host response window embedded in an EMR, displaying an acuity indicator for presenting an acuity score on the host response window, wherein the acuity score is an output of a machine learning model and the acuity score from the machine learning model determines a probability and level of a type of host response based on patient data, displaying one or more prognostic indicators including one or more prognostic values on the host response window, wherein the prognostic value is an output of the machine learning model and the prognostic value from the machine learning model determines a probability of an adverse event, identifying, one or more critical parameters in the patient data by comparing the one or more parameters in the patient data with a distribution of corresponding one or more parameters of a target population, and displaying a list of the parameters according to a ranking based on influence scores associated with the one or more parameters, and displaying an emphasis indicator drawing attention to one of the parameters.

Embodiment 29. An apparatus comprising one or more processors and one or more memory devices, wherein the one or more memory devices comprise instructions that, when executed by the processor, configure the one or more processors to perform operations comprising receiving, from a management platform, at least one electronic record associated with a patient, wherein the at least one electronic record comprises patient data, employing a machine learning model to generate an acuity score based on at least one of the parameters of the patient data, the acuity score representing probability and level a type of host response by the patient, employing a machine learning model to generate a prognostic value based on at least one of the parameters of the patient data, the prognostic value representing probability and level of an adverse event, identifying critical parameters in the patient data by comparing parameters in the patient data with a distribution of parameters of a target population, determining a ranking of the parameters according to an influence score associated with the critical parameters, and generating a dashboard GUI for displaying, on one or more client devices, the dashboard GUI comprising an acuity indicator displaying the acuity score on the dashboard GUI and specifying a risk category, a prognostic indicator displaying the prognostic value on the dashboard GUI and specifying a risk category, and a list displaying the parameters according to the ranking.

What is claimed is:

1. A system for generating an interactive dashboard graphical user interface (GUI) for displaying indicators based on one or more acuity values, associated with a risk category, the system comprising:
one or more processors; and
one or more memory devices, wherein the one or more memory devices comprise instructions that, when executed by the processor, configure the one or more processors to perform operations comprising:
transmitting a patient ID to a management platform;
receiving, from the management platform, at least one electronic record associated with a patient, the at least one electronic record comprising patient data;
employing a machine learning model to generate an acuity score based on the patient data, the acuity score representing probability and level of a type of host response by the patient;
employing a machine learning model to generate one or more prognostic values based on the patient data, the prognostic value representing a probability of an adverse event;
determining a ranking of the at least one parameter according to an influence score associated with the at least one parameter; and
generating a dashboard GUI for displaying, on or more client devices, the dashboard GUI comprising:
an acuity indicator displaying the acuity score and an associated risk category;
at least one prognostic indicator displaying the at least one prognostic value and one or more risk categories associated with the at least one prognostic value; and
a list displaying the parameters according to the ranking.

2. The system of claim 1, wherein the acuity score comprises a probability of the patient currently experiencing or developing the type of host response due to stimuli within a time period.

3. The system of claim 2, wherein the type of host response includes an undesirable host response.

4. The system according to any one of claims 2-3, wherein the stimuli includes at least one of infection, therapy, or trauma.

5. The system of claim 4, wherein the at least one of infection, therapy, or trauma includes sepsis.

6. The system of claim 2, wherein the time period is 24 hours or less.

7. The system according to any one of the preceding claims, wherein the adverse events comprise at least one of death, 30-day readmission, escalation to ICU, vasopressor administration, renal replacement therapy, extended length of stay, increased cost of patient stay, extracorporeal membrane oxygenation intervention, or mechanical ventilation.

8. The system according to any one of the preceding claims, wherein the dashboard GUI further comprises a workflow status displaying a treatment timetable identified by the machine learning model, wherein the treatment timetable includes at least one of order time, administration time, treatment time.

9. The system according to any of the preceding claims wherein the dashboard GUI comprises a timetable displaying selected parameters used by the machine learning model, wherein the selected parameters include at least one of a timetable displaying an order time, a blood draw time, a recorded time, and a result time.

10. The system according to any one of the preceding claims, wherein the dashboard GUI further comprises:
a notification based on the prognostic value and an output from the machine learning model; and
one or more interactive timers, wherein the one or more interactive times comprise a first timer displaying a time from a reference point and a second timer displaying a time left to complete treatment and diagnostic actions before violating care guidelines.

11. The system according to any one of the preceding claims, wherein the target population comprises patients suspected of having an infection.

12. The system of claim 11, wherein the infection includes sepsis.

13. The system according to any one of the preceding claims, wherein:
the dashboard GUI comprises an interactive population selector.

14. The system of claim 13, wherein the interactive population selector comprises a scatter plot and an area selection tool configured to an area of the scatter plot.

15. The system according to any one of the preceding claims, wherein the target population is based on pretest probability and patient location.

16. The system according to any one of the preceding claims, wherein the target population is defined by a second machine learning model, the second machine learning model being an unsupervised model.

17. The system according to any one of the preceding claims, wherein:
identifying the parameters comprises calculating a univariate distance score between the selected parameters and the parameters of the target population; and
determining the ranking comprises determining the ranking independently for each patient in the patient data and comparing the univariate score between the parameters.

18. The system according to any one of the preceding claims, wherein:
identifying the parameters comprises calculating individual parameters contributions using at least one of SHAP (SHapley Additive exPlanations) or Mahalanobis methods; and
determining the ranking comprises employing the at least one of SHAP or Mahalanobis methods to compare the parameters.

19. The system according to any one of claims 8-13, wherein:
the dashboard GUI further comprises an additive explanation bar plot;
the selected parameters comprise at least one of patient lab results, patient biomarker results, patient clinical parameters, derivative results, or patient trajectory information;
the timetable displayed on the dashboard GUI comprises interactive hide/show buttons; and
the timetable displays a result time and a value for each of the selected parameters.

20. The system according to any one of the preceding claims, wherein the dashboard GUI further comprises a checklist of treatment and diagnostic actions recommended by care guidelines for septic patients, the checklist comprising items for one or more of administration of antibiotics, ordering of blood cultures prior to antibiotics, measurement of serum lactate, administration of fluid resuscitation, or administration of vasopressors,
wherein the checklist displays a status of flow for each one of the items, the status of flow specifying at least one of physician order status, pharmacy approval status, administration of medication status, or full guidelines completed status.

21. The system according to any one of the preceding claims, wherein the dashboard GUI displayed is displayed embedded into a patient chart.

22. The system according to any one of the preceding claims, wherein employing the machine learning model comprises:
storing previously collected patient data from different target populations; and
returning a location associated with the patient data with reference to the target population.

23. The system according to any one of the preceding claims, wherein employing the machine learning model comprises:
performing an API call to a machine learning server, the API call comprising the patient data; and
receiving from the machine learning server the acuity score and the at least one prognostic value, the risk category of the acuity score, the one or more risk categories of the one or more prognostic values, the selected parameters, and the influence score of each parameter.

24. The system according to any one of the preceding claims, wherein:
the dashboard GUI is configured to be displayed on a mobile device associated with a healthcare professional.

25. The system according to any one of the preceding claims, wherein the operations further comprise:
coupling to one or more of a point of care diagnostic or a measurement device to the system; and
collecting a portion of the patient data directly from the point of care diagnostic or the measurement device.

26. The system according to any one of the preceding claims, wherein the operations further comprise:
training the machine learning model using supervised algorithms trained using one or more labels correlating to the types of host response defined by an output of an unsupervised algorithm.

27. The system according to any one of the preceding claims, wherein the risk category associated with the acuity score includes one of low, medium, high, or very high.

28. A computer implemented method for generating a dashboard GUI for displaying host response metrics, the method comprising:
coupling an analytics server with a management platform through a FHIR API;
generating a host response window embedded in an EMR;
displaying an acuity indicator for presenting an acuity score on the host response window, wherein the acuity score is an output of a machine learning model and the acuity score from the machine learning model determines a probability and level of a type of host response based on patient data;
displaying one or more prognostic indicators including one or more prognostic values on the host response window, wherein the prognostic value is an output of the machine learning model and the prognostic value from the machine learning model determines a probability of an adverse event;
identifying, one or more critical parameters in the patient data by comparing the one or more parameters in the patient data with a distribution of corresponding one or more parameters of a target population; and
displaying a list of the parameters according to a ranking based on influence scores associated with the one or more parameters; and
displaying an emphasis indicator drawing attention to one of the parameters.

29. An apparatus comprising:
one or more processors; and
one or more memory devices, wherein the one or more memory devices comprise instructions that, when executed by the processor, configure the one or more processors to perform operations comprising:
receiving, from a management platform, at least one electronic record associated with a patient, wherein the at least one electronic record comprises patient data;
employing a machine learning model to generate an acuity score based on at least one of the parameters of the patient data, the acuity score representing probability and level of a type of host response by the patient;
employing a machine learning model to generate a prognostic value based on at least one of the parameters of the patient data, the prognostic value representing probability and level of an adverse event;
identifying critical parameters in the patient data by comparing parameters in the patient data with a distribution of parameters of a target population;
determining a ranking of the parameters according to an influence score associated with the critical parameters; and
generating a dashboard GUI for displaying, on one or more client devices, the dashboard GUI comprising:
an acuity indicator displaying the acuity score on the dashboard GUI and specifying a risk category;
a prognostic indicator displaying the prognostic value on the dashboard GUI and specifying a risk category;
and
a list displaying the parameters according to the ranking.
